(19) United States
(12) Patent Application Publication
(10) Pub. No.: US 2017/0260210 A1
(43) Pub. Date: Sep. 14, 2017

(54) METHOD FOR PREPARING MARMYCIN A AND ANALOGUES THEREOF, AND ALSO USES THEREOF

(71) Applicants: Centre national de la recherche scientifique, Paris (FR); Université Pierre et Marie Curie (Paris 6), PARIS (FR)

(72) Inventors: Raphaël RODRIGUEZ, GIF SUR YVETTE (FR); Tatiana CANEQUE, GIF SUR YVETTE (FR); Filipe GOMES DA CRUZ, CHILLY MAZARIN (FR); Trang MAI, GIF SUR YVETTE (FR); Max MALACRIA, PARIS (FR); Giovanni MAESTRI, Mantova (IT)

(21) Appl. No.: 15/508,808

(22) PCT Filed: Sep. 1, 2015
(86) PCT No.: PCT/EP2015/069899

§ 371 Date: Mar. 3, 2017

(30) Foreign Application Priority Data
Sep. 5, 2014 (FR) 14 58364
Jan. 23, 2015 (FR) 15 50547

(51) Int. Cl. C07D 519/00; G01N 33/50; C07D 498/04; C07D 309/14; C07D 491/08

(52) U.S. Cl. CPC C07D 519/00; C07D 309/14; C07D 491/08; C07D 498/04; G01N 33/5091

(57) ABSTRACT

The present invention relates to a method for preparing marmycin A and analogues thereof, to novel marmycin A analogues, and also to the use of these compounds as an organelle marker and in pharmacy, in particular as antibiotics, anticancer agents and antimalarials.

The present invention is directed towards a method for preparing marmycin A and analogues thereof, novel analogues of marmycin A, and uses thereof as biomarkers in particular as organelle markers, and in pharmacy particularly as antibiotics, anticancer agents and antimalarials.

Marmycin A belongs to the family of angucyclines having antibacterial and cytotoxic properties. It is characterized by a polyaromatic structure including an anthraquinone repeating unit fused to an aminopyranose ring via a C-glycoside bond and N-glycoside bond on this same osidic group. Marmycin A therefore has a unique hexacyclic structure.

This natural molecule was isolated for the first time by Fenical et al. (W. Fenical, J. Nat. Prod. 2007, 70, 1406-1409) from cultures of an actinomycete derived from marine sediments of genus Streptomyces. This natural product is therefore only scarcely available in nature and is no longer commercially available. In addition, the total synthesis of this molecule, which would allow its pharmaceutical development, has not been carried out up until now.

There is therefore a need for a method to prepare marmycin A and the analogues thereof, including novel analogues of marmycin A.

It is the objective of the present invention to provide a method for the preparation via chemical synthesis of marmycin A and its analogues. The objective in particular is to provide total synthesis of Marmycin A and its analogues from commercial compounds. A further objective of the present invention is to provide novel analogues of marmycin A. It also sets out to provide compounds useful in pharmacy, in particular for the treatment of cancers, bacterial infections and/or malaria. A further objective of the present invention is to provide compounds which can be used as biomarkers, in particular as organelle biomarkers, especially as lysosome markers.

The present invention concerns a method to prepare a compound of formula (II):

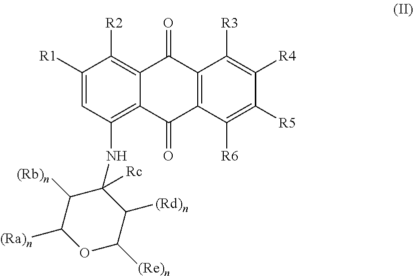

where R₁, R₂, R₃, R₄, R₅, R₆, Ra, Rb, Rc, Rd and Re are each independently an atom or group of atoms; n represent the number of Ra, Rb, Rd and Re radicals and are equal to 2;
said method comprising a coupling step B of a compound of formula (l):

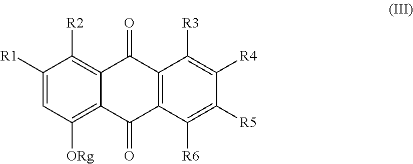

where the radicals R₁ to R₆ are such as defined for the compound of formula (II) and Rg is a reactive group with the NH₂ group of the formula (IV) compound, in particular a triflate group,
with a compound of formula (IV):

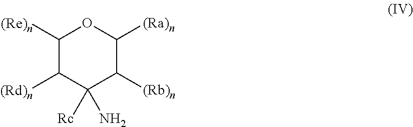

where the radicals Ra to Re and n are such as defined for the formula (II) compound, said coupling step being performed in the presence of a copper-containing compound.

The present invention, according to one variant, concerns a method to prepare a compound of formula (II):

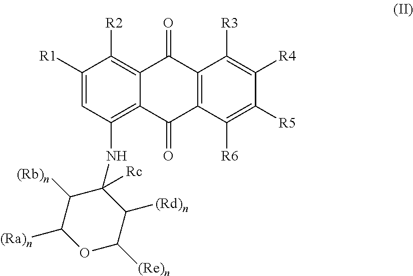

where:
R₁, R₂, R₃, R₄, R₅ and R₆ are each independently selected from the group formed by:
   H, OH, halogen, C(O)OH, ═O (═O is an oxygen atom bonded via a covalent double bond and the representation of the aromatic double bonds of the compounds is modified accordingly), (C₁-C₁₀)alkyl, O(C₁-C₁₀)alkyl, OC(O)(C₁-C₁₀)alkyl, (C₂-C₁₀)alkenyl, C(O)O(C₁-C₁₀)alkyl, NH₂, NH(C₁-C₁₀)alkyl, N[(C₁-C₁₀)alkyl]₂, NHC(O)(C₁-C₁₀)alkyl, N(C₁-C₁₀)alkyl-C(O)(C₁-C₁₀)alkyl, C(O)NH2, C(O)N(C₁-C₁₀)alkyl, C(O)N[(C₁-C₁₀)alkyl]₂, oses and epoxy groups, wherein said alkyls and/or said oses can be substituted; or
R₁ with R₂ and/or R₃ with R₄ and/or R₄ with R₅ and/or R₅ with R₆, together with the carbon atoms to which they are attached, form a (C₃-C₁₀)cycloalkyl or (C₆-C₁₀)aryl group, said cycloalkyl or aryl groups optionally being substituted and wherein at least one of the carbon atoms may optionally be replaced by a heteroatom, preferably by an oxygen atom;
the radicals Ra are each independently selected from the group formed by: H, OH, halogen, C(O)OH, ═O, (C₁-C₁₀)alkyl, O(C₁-C₁₀)alkyl, OC(O)(C₁-C₁₀)alkyl, (C₂-C₁₀)alkenyl, C(O)O(C₁-C₁₀)alkyl, NH₂, NH(C₁-C₁₀)alkyl, N[(C₁-C₁₀)alkyl]₂, NHC(O)(C₁-C₁₀)alkyl, N(C₁-C₁₀)alkyl-C(O)(C₁-C₁₀)alkyl, C(O)NH2, C(O)N(C₁-C₁₀)alkyl, C(O)N[(C₁-C₁₀)alkyl]₂ and the reactive groups allowing the formation of a C—C glycosidic bond, where said alkyls can be substituted;
the radicals Rb, Rc and Re are each independently selected from the group formed by: H, OH, halogen, C(O)OH, ═O, (C₁-C₁₀)alkyl, O(C₁-C₁₀)alkyl, OC(O)(C₁-C₁₀)alkyl, (C₂-C₁₀)alkenyl, C(O)O(C₁-C₁₀)alkyl, NH₂, NH(C₁-C₁₀)alkyl, N[(C₁-C₁₀)alkyl]₂, NHC(O)(C₁-C₁₀)alkyl, N(C₁-C₁₀)alkyl-C(O)(C₁-C₁₀)alkyl, C(O)NH2, C(O)N(C₁-C₁₀)alkyl and C(O)N[(C₁-C₁₀)alkyl]₂, where said alkyls can be substituted;
the radicals Rd are each independently selected from the group formed by: H, OH, halogen, C(O)OH, ═O, (C₁-C₁₀)alkyl, O(C₁-C₁₀)alkyl, OC(O)(C₁-C₁₀)alkyl, (C₂-C₁₀)alkenyl, C(O)O(C₁-C₁₀)alkyl, NH₂, NH(C₁-C₁₀)alkyl, N[(C₁-C₁₀)alkyl]₂, NHC(O)(C₁-C₁₀)alkyl, N(C₁-C₁₀)alkyl-C(O)(C₁-C₁₀)alkyl, C(O)NH2, C(O)N(C₁-C₁₀)alkyl, C(O)N[(C₁-C₁₀)alkyl]₂, (C₁-C₁₀)alkyl-COO⁻, methoxymethyl oxygen and the reactive groups allowing the formation of an O—C bond such as AcO, where said alkyls can be substituted;
n represent the number of Ra, Rb, Rd and Re radicals and are equal to 2; The subscript n of the compounds of the invention stresses that the radicals Ra, Rb, Rd and Re that are present may be the same or different.

Several attempts to obtain total synthesis of marmycin A have been carried out. However, total synthesis of this natural product has never been described to date, solely C-3-desmethyl-marmycin A having been fully synthesised (N. Maugel and B. B. Snider, Organic Letters, 2009, vol. 11, no. 21, 4926-4929 and A. Zhang et al., J. Org. Chem. 2009, 74, 6111-6119). However, C-3-desmethyl-marmycin A does not raise the same synthesis difficulties since it does not comprise any neopentyl amine unlike marmycin A. C-3-desmthyl-marmycin A does not contain the methyl group at position 3 on the ose. And compounds comprising alcohols or neopentyl amines such as marmycin A are known to have low reactivity.

The present inventors have discovered a novel method in fully unexpected manner allowing the total synthesis of marmycin A and analogues.

In particular, and surprisingly, the inventors have discovered that coupling B of the anthraquinone core with aminopyranose through the formation of a C—N glycosidic bond could be conducted in the presence of copper, e.g. in accordance with the principle of Ullmann coupling.

This reaction is most surprising since coupling of Buchwald-Hartwig type that is usual for this type of reaction (cf. Org. Synth. 2002, 78, 23 DOI: 10.15227/orgsyn.078.0023), conducted in the presence of palladium, does not allow obtaining of the C—N glycosidic bond in the present case.

Coupling B according to the invention notably allows a good yield to be obtained, preferably at least 30% of the formula (II) compound of the invention.

In addition, for the formation of anthraquinone, the inventors surprisingly carried out coupling A of Diels-Alder type in a single reactor. The Diels-Alder reaction is normally carried out in several reactors (J. Org. Chem., 2007, 72 (16), pp 6116-6126). The method of the invention is therefore simplified compared with conventional Diels-Alder reactions.

At a third step of the method of the invention, the inventors have unexpectedly developed a cyclisation step C allowing a pentacyclic structure to be obtained. Two alternatives can be used to perform this cyclisation step.

According to a first cyclisation alternative Ca, a glycosidic C—C bond is formed with intramolecular cyclisation. Surprisingly, this cyclisation is particularly possible in the presence of tetrafluoroboric acid. This first alternative is particularly suitable for preparing marmycin A and analogues thereof, in particular the formula (Ia) compounds of the invention.

According to a second cyclisation alternative Cb, an O—C bond is formed with intramolecular cyclisation. Surprisingly, this cyclisation is particularly possible in the presence of a base for the preparation of oxamarmycin A and derivatives thereof, in particular to obtain the formula (Ib) compounds of the invention.

A fourth addition step, allowing the formation of novel analogues of marmycin A, and in particular the formula (IaD) compounds of the invention, has also been developed by the inventors.

The present invention therefore particularly has the advantage of allowing the total synthesis of marmycin A or analogues, with starting reagents that are commercially available. Since this method can implemented on industrial level, it therefore allows marmycin A and analogues to be obtained in sufficient quantity for their pharmaceutical development and marketing. With the method of the invention it is notably possible to obtain a sufficient yield of marmycin A or analogues.

It has also been possible to discover novel analogues, in particular oxamarmycin A and derivatives thereof.

The inventors have also evidenced the properties of the compounds of the invention for the marking of organelles contained in the cytosol in particular e.g. lysosomes, and anticancer, antibiotic and antimalarial properties of the compounds of the invention.

The inventors have discovered that the compounds of the invention do not accumulate in cell nuclei but in cell organelles such as lysosomes.

In particular, and without wishing to be bound by any theory, it would seem that marmycin A and analogues thereof accumulate in organelles e.g. lysosomes and produce Reactive Oxygen Species—ROS. These reactive oxygen species appear to induce permeabilization of organelles, in particular of the lysosomal membrane, possibly leading to release of cathepsins and other hydrolases in the cytosol. This permeabilization in some cases could also lead to permeabilization of the outer membrane of mitochondria. Permeabilization of the membranes of these organelles could then induce cell apoptosis (cf. Oncogene (2008) 27, 6434-6451).

Surprisingly, the inventors have discovered that the compounds of formula (IaD) have higher anti-proliferative activity than marmycin A.

Even more surprisingly, the inventors have discovered that covalent grafting between marmycin A and its analogues and a heterocyclic precursor compound of free radicals allows the compounds obtained to have anti-proliferative action of synergic type, in particular the formula (IaD) compounds of the invention. The anti-proliferative activity of these compounds is higher than the activity of marmycin A and the heterocyclic compounds taken alone, and higher than the anti-proliferative activity of a combination thereof.

The present invention therefore concerns pharmaceutical compositions comprising the compounds of the invention, and the compounds of the invention for therapeutic uses thereof or as medicinal product.

The present invention therefore concerns a therapeutic treatment method. These applications are described in more detail in the descriptions of the applications of the compounds of the invention.

### Definitions

In the present invention, the term <<(C₁-C₁₀)alkyl>> designates saturated aliphatic hydrocarbon radicals, straight-chain or branched, having 1 to 10 carbon atoms, preferably 1 to 5 carbon atoms. A branched chain means that one or more lower alkyl groups such as methyl, ethyl or propyl, are linked to the main alkyl straight chain. The preferred alkyl groups are methyl, ethyl, propyl or isopropyl, in particular methyl. The term <<alkyl>> comprises substituted alkyl groups wherein on or more hydrogen atoms are substituted by another atom or group of atoms.

The term <<(C₁-C₁₀)alkylene>> designates a divalent (C₁-C₁₀)alkyl radical such as defined above.

The term <<(C₂-C₁₀)alkenyl>> designates an alkyl group having 2 to 10 carbon atoms, preferably 2 to 5 carbons atoms, unsaturated i.e. comprising at least one carbon-carbon double bond, and which may be straight-chain or branched. As an example of an alkenyl group, particular mention can be made of ethenyl, propenyl, n-butenyl, i-butenyl, 3-methylbut-2-enyl, or n-pentenyl. The term <<alkenyl>> comprises substituted alkenyl groups wherein one or more hydrogen atoms are substituted by another atom or group of atoms.

The term <<(C₃-C₁₀)cycloalkyl>> means a mono- or multicyclic non-aromatic ring system having 3 to 10 carbon atoms, preferably 5 to 6 carbon atoms, wherein each substitutable atom of the ring is optionally substituted. As an example of monocyclic cycloalkyl, particular mention can be made of cyclopentyl, cyclohexyl or cycloheptyl. The term <<cycloalkyl>> comprises the substituted cycloalkyl groups wherein one or more hydrogen atoms are substituted by another atom or group of atoms.

The term <<aryl>> designates an aromatic monocyclic or multicyclic ring system having 6 to 10 carbon atoms wherein each substitutable atom of the ring is optionally substituted by another atom or group of atoms. As examples of aryl groups particular mention can be made of phenyl, naphthalene and anthracene.

The term <<(C₃-C₂₀)heterocycle>> designates a non-aromatic mono- or multicyclic ring system having 3 to 20 carbon atoms, wherein each substitutable atom of the ring is optionally substituted and wherein at least one of the carbon atoms is replaced by a heteroatom, preferably by an oxygen atom. Preferably, the heterocycle is a multicyclic non-aromatic ring system, e.g. quadricyclic comprising at least one organic peroxide function (—C—O—O—C).

The term <<ose>> (or monosaccharide) designates a carbohydrate monomer. Oses have at least 3 carbon atoms and particularly comprise trioses, tetroses, pentoses, hexoses, deoxy-hexoses (fucose or rhamnose), heptoses and nonoses. The preferred oses of the invention are hexoses such as pyranose, allose, altrose, galactose, glucose, gulose, idose, mannose, talose, fructose, sorbose and tagatose, preferably pyranose. According to one particular embodiment, by <<ose>> are meant the formula (IV) compounds of the invention.

The term <<epoxy>> designates the groups of following formula:

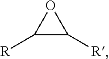

where R and R′ are each independently selected from among the groups (C₁-C₁₀)alkyl, O(C₁-C₁₀)alkyl, OC(O)(C₁-C₁₀)alkyl, (C₂-C₁₀)alkenyl, C(O)O(C₁-C₁₀)alkyl, NH(C₁-C₁₀)alkyl and N[(C₁-C₁₀)alkyl]₂, wherein said alkyls may be substituted.

The term <<halogen>> designates a chlorine, bromine, iodine or fluorine atom.

The term <<heteroatom>> designates an atom selected from among O, N, S or P, preferably O or N.

By <<group reactive with a NH₂ group of the formula (IV) compound>> is meant any group allowing the formation of a C—N bond between the formula (III) compound and the formula (IV) compound of the invention, in particular a triflate group.

By <<group reactive with an OH group of the formula (Ie) compound>> is meant any group allowing the formation of a covalent bond e.g. of O—C type between the formula (Ia) compound and the spacer group L, in particular a C(O)OH or OH group.

The term <<protective group>> designates a group protecting functional chemical groups, in particular alcohol functions against undesirable reactions during synthesis reactions. Examples of protective groups are given in T. W. Greene and P. G. M. Wuts, Protective Groups in Organic Synthesis, 3rd edition, John Wiley & Sons, New York (1999), and are known to persons skilled in the art. Particular mention is made of CH₃—C(O).

By ester function is meant a C—C(O)—O—C group. By etheroxide function is meant a C—O—C group. By carbonate function is meant a C—O—C(O)—O—C group. By carbamate function is meant a C—NH—C(O)—O—C group.

### Method to Prepare Compounds of Formula (II)

### Coupling Step B

According to one embodiment, the method to prepare a formula (II) compound of the invention is characterized in that the coupling step B is conducted in the presence of a cooper-containing compound of formula Cu—X, X being selected from the group formed by Cl, Br, I, SO₄ and OAc, and preferably of formula Cu—I.

According to one embodiment, the coupling step B is conducted in the presence of 5 mol % to 25 mol %, preferably between 10 mol % and 20 mol %, of copper-containing compound. Preferably, the coupling step B is conducted in the presence of 10 mol % or 20 mol % Cu—X. According to one embodiment, said coupling step B is conducted in the presence of 1 to 6 eq. of formula (IV) compound such as defined above, preferably 2, 3, 4 or 5 eq.

According to one embodiment, said coupling B is conducted in the presence of a base e.g. K₂CO₃, preferably in the presence of a solvent e.g. benzene, xylene, toluene or acetonitrile.

According to one embodiment, said coupling step B is conducted in the presence of base e.g. K₂CO₃, in an amount of between 1 eq. and 4 eq., and preferably 2 eq.

According to one embodiment, said coupling step B is conducted in the presence of a solvent such as toluene or acetonitrile, preferably in the presence of toluene.

According to another embodiment, said coupling step B is conducted in the presence of Cu—I as catalyst, K₂CO₃ as base and toluene as solvent, preferably under reflux.

According to one embodiment, said coupling step B is conducted at a temperature of between 75° C. and 170° C., preferably between 130° C. and 170° C., more preferably between 140° C. and 160° C. According to one embodiment, said coupling step B is conducted at a temperature of 140° C., 150° C. or 160° C.

According to another embodiment, said coupling step B is conducted for at least 24 h, preferably between 24 h and 72 h.

According to one particular embodiment, the reaction is conducted in the presence of 20 mol % Cu—I, 2 eq. K₂CO₃ and toluene, at 160° C.

### Coupling Step A

According to one embodiment, the method of the invention also comprises a coupling step A of a compound of formula (V):

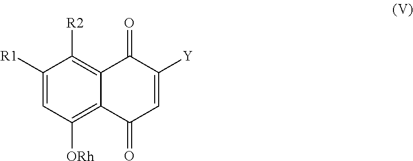

where R₁ and R₂ are such as defined for formula (II), Y being a reactive group with the carbon atom carrying the group R₃ of the formula (VI) compound and Rh being a protective group, preferably a CH₃—C(O) group, with a compound of formula (VI):

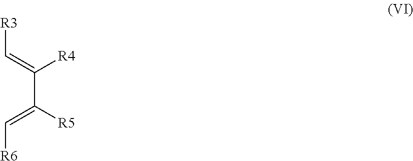

to obtain a compound of formula (III′):

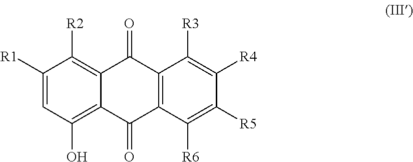

in the presence of an organic solvent, preferably toluene or xylene, preferably in the presence of toluene.

According to one embodiment, Y is selected by F, Cl, Br and I, preferably Br.

According to one embodiment, the reaction is performed under reflux.

According to one embodiment, said coupling step A is conducted at a temperature between 80° C. and 120° C.

According to one particular embodiment, the coupling step A comprises the following steps:
i) coupling step A such as described above;
ii) evaporation of the medium, preferably in a bath at a temperature between 50° C. and 70° C., e.g. 60° C.;
iii) adding at least one alcohol, preferably methanol or ethanol, preferably methanol;
iv) adding a base e.g. potassium carbonate, and deprotection of the alcohol function with removal of Rh; and
v) obtaining the formula (III′) compound such as defined above.

Preferably, step iii) is conducted under agitation for at least 1 h. According to one embodiment, all the steps i) to iv) of coupling step A are performed in a single reactor (one pot).

According to one embodiment, the method of the invention further comprises an additional activation step of the formula (III′) compound by substitution of the hydrogen atom of the alcohol by a reactive group Rq with the NH₂ group of the formula (IV) compound of the invention, e.g. —SO₂—CF₃ typically obtained by reaction with triflic anhydride of formula F₃C—SO₂—O—SO₂—CF₃, to obtain the following compound of formula (III):

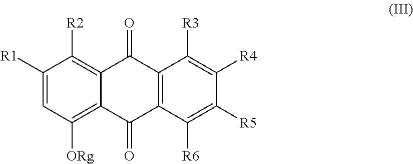

According to one embodiment, this additional activation step is performed in the presence of a base, in particular an amine base such as triethylamine, preferably in the presence of an apolar solvent e.g. dichloromethane.

According to one embodiment, said activation reaction is conducted at a temperature between −65° C. and −85° C.

### Method to Prepare Compounds of Formulas (Ia) and (Ib)

### Cyclisation Step C

According to one embodiment, the method of the invention also comprises a cyclisation step C of said formula (II) compound, to form a pentacyclic structure.

According to one particular embodiment, the method of the invention is a method to prepare a compound of following formula (Ia):

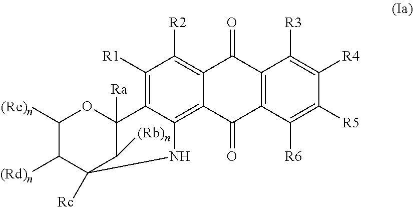

where the radicals R₁ to R₆ and Ra to Re are such as defined for the formula (II) compounds, said method comprising a cyclisation step Ca by C—C glycosylation of a formula (II) compound such as obtained in the invention, for example in the presence of HBF₄ to form the formula (Ia) compound.

According to one particular embodiment, the cyclisation step Ca is performed in the presence of HBF₄ OEt₂ or HBF₄ e.g. at 50% m/m in an aqueous solution. According to one particular embodiment, the cyclisation step Ca is conducted with an amount of HBF₄ OEt₂ of between 0.5 eq. and 5 eq., and more particularly 1, 2 or 3 eq. of HBF₄ OEt₂.

According to another embodiment, the cyclisation step Ca is conducted in the presence of 50% to 70% m/m of HBF₄ in aqueous solution, preferably 60% m/m of HBF₄ in aqueous solution.

According to one embodiment, the reaction is conducted in the presence of acetonitrile, preferably under reflux. Preferably, the reaction is conducted for at least 3 h, preferably between 4 h and 24 h.

According to one embodiment, the cyclisation step Ca is performed indirectly by reacting a formula (II) compound such as defined above with HBF₄, then reacting the reaction product in the presence of HBF₄ and acetonitrile, preferably under reflux, to obtain a compound of formula (Ia) of the invention.

According to another embodiment, the cyclisation step Ca is performed directly, by reacting a formula (II) compound in the presence HBF₄ or HCl and acetonitrile, preferably under reflux.

According to another particular embodiment, the method of the invention is a method to prepare compounds of following formula (Ib):

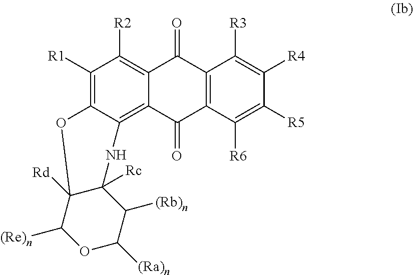

where the radicals R₁ to R₆ and Ra to Re are such as defined for the formula (II) compounds, and comprising a cyclisation step Cb by forming an O—C bond of a formula (II) compound such as obtained in the invention, in a basic medium, preferably in the presence of NaH to form the compound of formula (Ib).

According to one embodiment, at least one base capable of deprotonating an alcohol is used. Preferable use is made of NaH.

According to one embodiment, the cyclisation step Cb is performed in the presence of dichloromethane. According to another embodiment, the cyclisation reaction Cb is conducted at a temperature between −20° C. and 0° C., and preferably between −5° C. and 0° C., more preferably at 0° C.

According to one particular embodiment, the method to prepare the formula (Ia) compounds of the invention comprises the following steps:
i) coupling step A according to the invention,
ii) coupling step B according to the invention, and
iii) cyclisation step Ca according to the invention.

According to another particular embodiment, the method to prepare the formula (Ib) compounds of the invention comprises the following steps:
i) coupling step A according to the invention,
ii) coupling step B according to the invention,
iii) cyclisation step Cb according to the invention.

### Method to Prepare Compounds of Formula (IaD)

### Addition Step D

According to one embodiment, the method of the invention also comprises an addition step D.

According to one embodiment, the addition step D is selected from the group formed by steps of esterification, etherification, carbonate formation and carbamate formation. These steps are known to persons skilled in the art and it is within their reach to determine suitable operating conditions. Preferably, addition step D is esterification.

According to one particular embodiment, esterification is conducted at ambient temperature e.g. at between 20° C. and 25° C. Preferably, esterification is conducted in the presence of a solvent e.g. in dichloromethane. According to one particular embodiment, the esterification reaction is carried out in the presence of 4-dimethylaminopyridine and/or N,N′-dicyclohexylcarbodiimide. According to one embodiment, the reaction product of formula (IaD) is purified under reduced pressure e.g. at between 5 mmHg and 15 mmHg, e.g. 10 mmHg.

According to one particular embodiment, the method of the invention is a method to prepare a compound of formula (IaD) such as defined below, said method comprising an addition step D of a compound of formula (Ia):

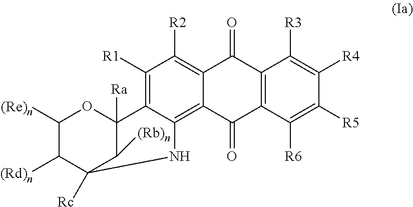

where the radicals R₁ to R₆, Ra to Re and n are such as defined in the invention and where at least one of the radicals Ra to Re is an OH group, preferably a radical Rd; with a compound of following formula (Ie):

Rx-L-Rz (Ie)

where:
Rx is a reactive group allowing reaction with an OH function, preferably a C(O)OH or OH group;
L is a group of atoms called a ((spacer group); and
Rz is an optionally substituted (C₃-C₂₀)heterocycle;
said addition step D resulting in the formation of a compound of following formula (IaD):

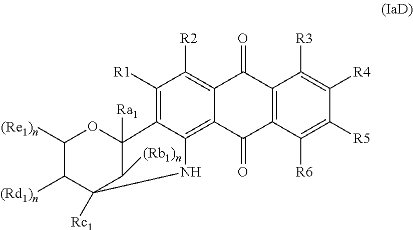

where the radicals R₁ to R₆ and n are such as defined in the invention and where the radicals Ra1 to Re1 have the same definitions as the radicals Ra to Re respectively defined for formula (Ia),
among which at least one of the radicals Ra1 to Re1, preferably Rd1, represents the group resulting from the reaction between the OH group and Rx, covalently bonded to L-Rz.

According to one embodiment, Rx is a C(O)OH group.

According to one embodiment, the spacer group L is a (C₁-C₁₀)alkylene able to be substituted and/or to comprise one or more heteroatoms selected from among O, N and S, and/or one or more ester, ether, carbonate and carbamate functions.

According to one embodiment, the spacer group L is a (C₁-C₁₀)alkylene able to be substituted and/or to comprise at least one function selected from the group formed by secondary amines, tertiary amines, esters, ether oxides, carbonates and carbamates, preferably esters. According to one particular embodiment, the spacer group L is a polyethylene glycol.

According to one particular embodiment, the spacer group L bonded to the group resulting from the reaction between the OH group and Rx is selected from the group formed by:

—O—C(O)—(C₁-C₁₀)alkylene-, —O—CH₂—(C₁-C₁₀)alkylene-, —O—C(O)—O(C₁-C₁₀)alkylene- and —O—C(O)—NH—(C₁-C₁₀)alkylene, where the (C₁-C₁₀)alkylenes may be substituted and/or comprise one or more heteroatoms selected from among O, N and S, and/or one or more ester, ether, carbonate and carbamate functions.

According to one embodiment, the optionally envisaged substituent(s) for the above (C₁-C₁₀)alkylene groups are selected from the group formed by OH, halogen, C(O)OH, ═O, (C₁-C₁₀)alkyl, O(C₁-C₁₀)alkyl, OC(O)(C₁-C₁₀)alkyl, (C₂-C₁₀)alkenyl, C(O)O(C₁-C₁₀)alkyl, NH₂, NH(C₁-C₁₀)alkyl and N[(C₁-C₁₀)alkyl]₂.

According to one particular embodiment, the spacer group L bonded to the group resulting from the reaction between the OH group and Rx represents a —O—C(O)—(C₁-C₅)alkylene-C(O)O— group.

According to one embodiment, Rz is a fused, optionally substituted multicyclic heterocycle, preferably a fused, optionally substituted quadricycle. According to one particular embodiment, Rz is a heterocycle of following formula, optionally substituted:

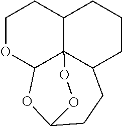 [SMILES: C1CC2CCOC3OC4CCC(C1)C23OO4]

Preferably, Rz is artemisinin, of formula:

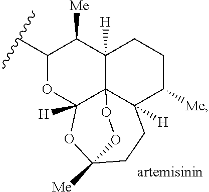

where [symbol] represents the bond with the spacer group L.

According to one embodiment, Rz is a (C₃-C₂₀)heterocycle optionally substituted by at least one substituent selected from the group formed by OH, halogen, C(O)OH, ═O, (C₁-C₁₀)alkyl, O(C₁-C₁₀)alkyl, OC(O)(C₁-C₁₀)alkyl, (C₂-C₁₀)alkenyl, C(O)O(C₁-C₁₀)alkyl, NH₂, NH(C₁-C₁₀)alkyl and N[(C₁-C₁₀)alkyl]₂, preferably by at least one substituent selected from among ═O and (C₁-C₁₀)alkyls such as methyl.

According to one particular embodiment, the addition step D is an esterification step of marmycin A:

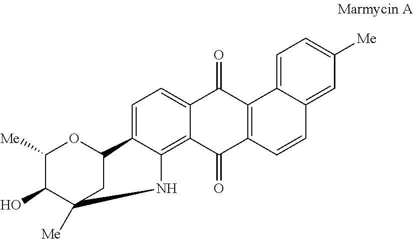

with artesunate of following formula:

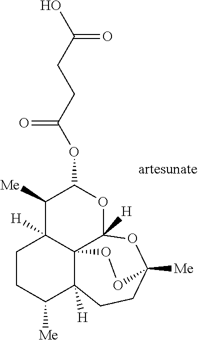

to form a compound of following formula (IaD):

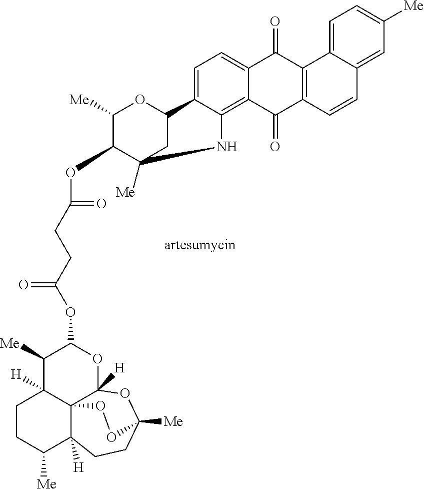

According to one particular embodiment, the method to prepare formula (IaD) compounds of the invention comprises the following steps:
i) coupling step A according to the invention,
ii) coupling step B according to the invention,
iii) cyclisation step Ca according to the invention, and
iv) addition step D according to the invention.

### Compounds of Formulas (Ia), (Ib), (IaD), (II), (III), (III′), (IV), (V) and (VI)

The particular embodiments below apply to the compounds of formulas (la), (Ib), (IaD), (II), (III), (III′), (IV), (V) and (VI) of the invention.

According to one variant, when at least one of the radicals Ra is selected from among the reactive groups allowing the formation of a glycosidic C—C bond, the radicals Rd are each independently selected from the group formed by: H, OH, halogen, C(O)OH, ═O, (C₁-C₁₀)alkyl, O(C₁-C₁₀)alkyl, OC(O)(C₁-C₁₀)alkyl, (C₂-C₁₀)alkenyl, C(O)O(C₁-C₁₀)alkyl, NH₂, NH(C₁-C₁₀)alkyl, N[(C₁-C₁₀)alkyl]₂, (C₁-C₁₀)alkyl-COO⁻ and methoxymethyloxygen, wherein said alkyls may be substituted.

According to a second variant, when at least one of the radicals Rd is selected from the reactive groups allowing the formation of an O—C bond such as AcO, the radicals Ra are each independently selected from the group formed by: H, OH, halogen, C(O)OH, ═O, (C₁-C₁₀)alkyl, O(C₁-C₁₀)alkyl, OC(O)(C₁-C₁₀)alkyl, (C₁-C₁₀)alkenyl, C(O)O(C₁-C₁₀)alkyl, NH₂, NH(C₁-C₁₀)alkyl and N[(C₁-C₁₀)alkyl]₂, wherein said alkyls may be substituted.

According to one particular embodiment, the reactive groups allowing the formation of a glycosidic C—C bond are selected from among OH and the O(C₁-C₁₀)alkyl groups, preferably OH and OMe.

The embodiments below apply to the groups Ra to Re of the compounds of formulas (la), (Ib), (II) and (IV), and Ra1 to Re1 when present i.e. when the compounds are formula (IaD) compounds.

According to one embodiment, Ra and Ra1 are OH or O(C₁-C₄)alkyl, e.g. methoxy.

According to one embodiment, Rb and Rb1 are H.

According to one embodiment, Rc and Rc1 are a (C₁-C₁₀)alkyl, preferably a (C₁-C₄)alkyl. According to one embodiment, Rc and Rc1 are a methyl.

According to one embodiment, Rd and Rd1 are OH or a methoxymethyloxygen.

According to one embodiment, Rd and Rd1 are not an AcO group. According to one embodiment, Rd and Rd1 are not an OH group.

According to one embodiment, Rb and Rb1 are H and Rc and Rc1 are a methyl.

According to one embodiment, Rb is H, Rc is a methyl and Re is a methyl.

According to one embodiment, Rb1 is H, Rc1 is a methyl and Re1 is a methyl.

According to one embodiment, R₁, R₂, R₅ and R₆ are H. According to one embodiment, R₁, R₅ and R₆ are H and R₂ is a halogen, preferably Cl, or H.

According to one embodiment, R₃ and R₄ together with the carbons to which they are attached are a (C₆-C₁₀)aryl, preferably an optionally substituted phenyl; According to one embodiment, R₃ and R₄, together with the carbon atoms to which they are attached, form a phenyl or cyclohexyl substituted by at least one substituent selected from the group formed by OH, methyl, ═O.

Preferably, said phenyl or cyclohexyl is substituted by one, two or three substituents each independently selected from among OH, CH₃ and ═O.

According to one embodiment, when R₃ and R₄ together form a phenyl, said phenyl is not substituted by a methyl.

According to one embodiment, the oses, cycloalkyls and aryls of the radicals R₁ to R₆ can be substituted at least once by a substituent selected from the group formed by OH, halogen, C(O)OH, ═O, (C₁-C₁₀)alkyl, O(C₁-C₁₀)alkyl, OC(O)(C₁-C₁₀)alkyl, (C₂-C₁₀)alkenyl, C(O)O(C₁-C₁₀)alkyl, NH₂, NH(C₁-C₁₀)alkyl, N[(C₁-C₁₀)alkyl]₂, and

the alkyls contained in the radicals Ra, Rb, Rc, Rd and Re and Ra1, Rb1, Rc1, Rd1 and Re1 are substituted by at least one substituent selected from the group formed by OH, halogen, C(O)OH, ═O, O(C₁-C₁₀)alkyl, OC(O)(C₁-C₁₀)alkyl, (C₂-C₁₀)alkenyl, C(O)O(C₁-C₁₀)alkyl, NH₂, NH(C₁-C₁₀)alkyl, N[(C₁-C₁₀)alkyl]₂.

According to one embodiment, the oses, cycloalkyls and aryls of the radicals R₁ to R₆ can be substituted at least once by a substituent selected from the group formed by CH₃, OH, ═O, COOH, COMe and NMe₂, preferably by Me, and the alkyls included in the radicals Ra, Rb, Rc, Rd and Re and Ra1, Rb1, Rc1, Rd1 and Re1 are substituted by at least one substituent selected from the group formed by OH, ═O, COOH, COMe, NMe₂.

The present invention also concerns a formula (II) compound, able to be obtained with the method of the invention.

The present invention also concerns a formula (Ia) compound able to be obtained with the method of the invention.

The present invention also concerns a formula (Ib) compound able to be obtained with the method of the invention.

The present invention also concerns a formula (IaD) compound able to be obtained with the method of the invention.

The present invention also concerns a compound of following formula (II):

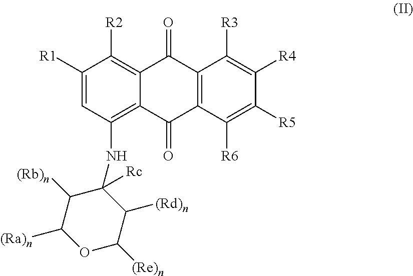

where the radicals R₁ to R₆, Ra to Re and n are such as defined in the invention.

According to one embodiment, Rc is a (C₁-C₁₀)alkyl, preferably a methyl.

According to one embodiment, the invention concerns a formula (II) compound such as defined above, with the exception of the compounds where R₃ and R₄ together with the carbon atoms to which they are attached form a phenyl group.

The present invention concerns a compound of following formula (Ia):

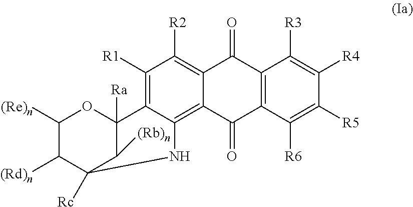

where the radicals R₁ to R₆, Ra to Re and n are such as defined in the invention, with the exception of the compounds where R₃ and R₄, together with the carbon atoms to which they are attached, form a phenyl group, and
with the exception of the following compounds:

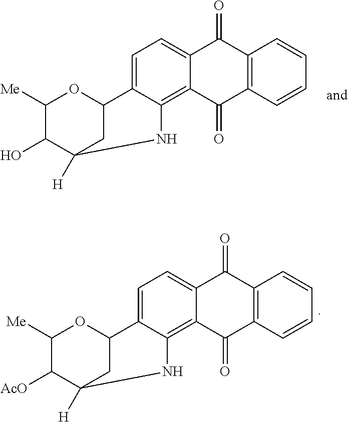 [SMILES: CC1OC2CC(Nc3c2ccc2c3C(=O)c3ccccc3C2=O)C1O ; "and" ; CC(=O)OC1C2CC(OC1C)c1ccc3c(c1N2)C(=O)c1ccccc1C3=O]

The present invention concerns a compound of following formula (Ib):

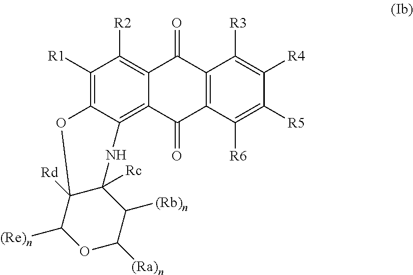

where the radicals R₁ to R₆, Ra to Re and n are such as defined in the invention.

According to one embodiment, the compounds of formulas (la) and/or (Ib) have the following configuration:

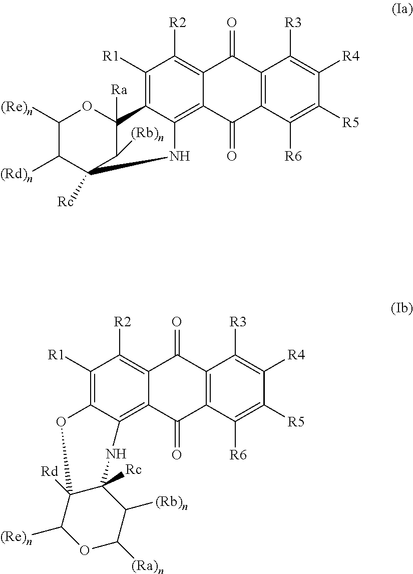

The present invention also concerns the following specific compounds:

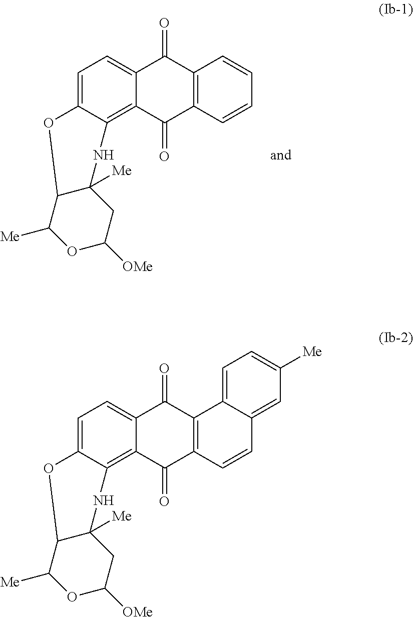 [SMILES: COC1CC2(C)Nc3c(ccc4c3C(=O)c3ccccc3C4=O)OC2C(C)O1 ; "and" ; COC1CC2(C)Nc3c(ccc4c3C(=O)c3ccc5cc(C)ccc5c3C4=O)OC2C(C)O1]

According to one embodiment, said compounds have the following formulas:

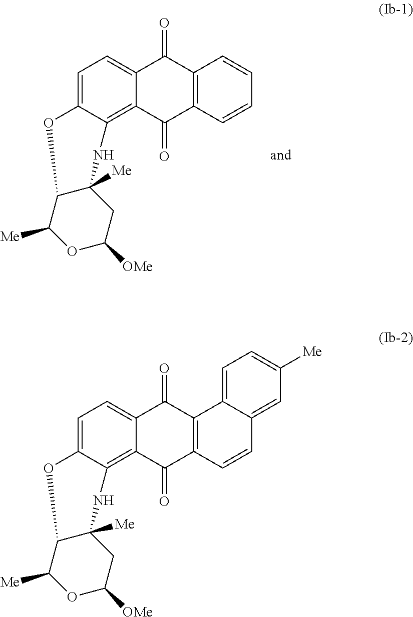 [SMILES: CO[C@@H]1C[C@@]2(C)Nc3c(ccc4c3C(=O)c3ccccc3C4=O)O[C@H]2[C@H](C)O1 ; "and" ; CO[C@@H]1C[C@@]2(C)Nc3c(ccc4c3C(=O)c3ccc5cc(C)ccc5c3C4=O)O[C@H]2[C@H](C)O1]

The present invention concerns a compound of following formula (IaD):

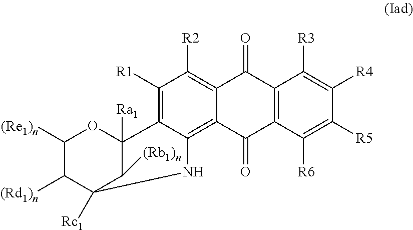

where the radicals R₁ to R₆, Ra1 to Re1 and n are such as defined in the invention.

The present invention concerns a compound of following formula (IaD):

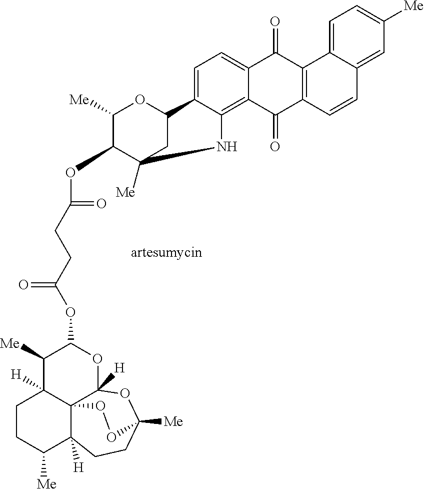

According to one particular aspect, the invention concerns the non-natural synthetic compounds of formula (Ia), (Ib) or (IaD). According to another aspect, the invention concerns the compounds of formulas (la) and (Ib) in crystalline form, and more particularly marmycin A in crystalline form referenced under number CCDC1015494 (Cambridge Cristallography Data Center).

The present invention also concerns the following specific, non-natural synthetic compound:

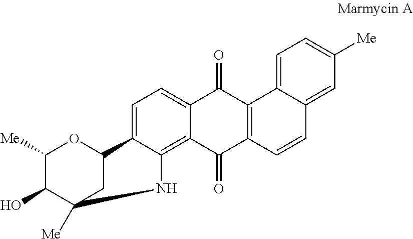

### Uses of Compounds of Formula (Ia), (Ib), (IaD) and (II)

The present invention also concerns a compound of formula (Ia), (Ib), (IaD) or (II) for use thereof in the treatment and/or prevention of a cancer, bacterial infection and/or malaria. Marmycin A is especially known to have cytotoxic properties on human breast, prostate, lung, colon, ovarian cancer lines and leukaemia (W. Fenical, J. Nat. Prod. 2007, 70, 1406-1409).

Marmycin A and analogues thereof accumulate in organelles such as lysosomes and produce Reactive Oxygen Species—ROS therein. These reactive oxygen species appear to induce permeabilization of the membrane of organelles. This permeabilization would then induce cell apoptosis (cf. Oncogene (2008) 27, 6434-6451). Yet permeabilization of the lysosomal membrane in particular is a process that is perturbed in cancer cells. The compounds of the invention are therefore particularly useful for the treatment of cancers.

By <<cancers>> are meant solid tumours or “liquid” tumours (also called haematological cancers) and/or their metastases. By “metastases” are meant secondary malignant tumours formed by migration of cancer cells to another region other than the region of the starting malignant tumour. Haematological cancers particularly comprise myeloma, lymphoma and leukaemia.

More particularly, the compounds of formula (Ia), (Ib), (IaD) or (II) of the invention are useful for the treatment and/or prevention of a cancer selected from among leukaemia, cancer of the colon, ovaries, breast, prostate, uterus (in particular the cervix), lung, bladder, brain, stomach, pancreas, liver, intestines, head, neck and skin, preferably breast, prostate, lung, colon, ovarian cancer and leukaemia.

Advantageously, one or more compounds of formula (Ia), (Ib), (IaD) or (II) of the invention are used in a method to treat and/or prevent a resistant cancer, and in particular a cancer having cancerous stem cells. By <<resistant cancers>> are particularly meant cancers resistant to conventional therapies (e.g. with taxol). For example, this may be a breast cancer resistant to conventional therapies. According to one variant, the invention concerns a treatment for breast cancer with resistant cancerous stem cells expressing the CD44 markers and under-expressing CD24 (CD44^{high}/CD24^{low}). According to one variant, the invention concerns a treatment for cancer with resistant cancerous cells expressing <<Human telomerase reverse transcriptase>> (hTERT) for example. According to one variant, the invention concerns a treatment for cancer with resistant cancerous stem cells expressing the markers CD133 and/or ALGH (CD133⁺ and/or ALGH⁺) (e.g. Glioblastoma multiforme (GBM)). According to one variant, the compounds (Ia), (Ib), (IaD) or (II) of the invention are used for therapeutic second-line treatment of a cancer.

Advantageously, one or more compounds of formula (Ia), (Ib), (IaD) or (II) of the invention are used in a method to treat and/or prevent a cancer having cancerous stem cells.

The following table gives types of solid malignant tumours and a non-exhaustive list of cell surface markers of their cancerous stem cells (Laurie E Ailles and Irving L Weissman, Cancer stem cells in solid tumors. Current Opinion in Biotechnology 2007, 18:460-466; Tirino V, Desiderio V, Paino F, De Rosa A, Papaccio F, La Noce M, Laino L, De Francesco F, Papaccio G. Cancer stem cells in solid tumors: an overview and new approaches for their isolation and characterization. FASEB J. 2013 January; 27(1):13-24):

| Type of tumour | CSC surface phenotype |
|---|---|
| Breast cancer | CD44⁺CD24^{-/low} |
| Glioblastoma | CD133⁺ |
| Melanoma | CD20⁺ |
| Prostate cancer | CD44⁺/α₂β₁^{hi}/CD133⁺ |
| Ovarian cancer | CD44⁺ |
| Gastric cancer | CD44⁺ |
| Pancreatic cancer | CD44⁺ EpCAM⁺ CD24⁺ |
| Lung cancer | CD133⁺ |
| Colon cancer | CD133⁺ |
| | CD44⁺ EpCAM⁺ CD166⁺ |
| Hereditary nonpolyposis | CD44⁺ |
| colorectal cancer/Head and Neck | |
| squamous cell carcinoma—HNSCC | |
| Osteosarcoma | CD133⁺, CD117⁺, Stro-1⁺ |
| Chondrosarcoma | CD133⁺ |
| Synovial sarcoma | CD133⁺ |
| Ewing's sarcoma | CD133⁺ |
| Rhabdomyosarcoma | CD133⁺ |
| Multiple myeloma | CD138⁻ |

According to one variant, the invention concerns treatment for a tumour of the haematopoietic system having cancerous stem cells expressing the markers CD34 and under-expressing CD38 (CD34+/CD38−).

By <<bacterial infections>> are meant pathologies due to contamination of the body with bacteria.

By <<malaria>> is meant the potentially fatal infectious disease due to a parasite of the genus Plasmodium such as Plasmodium falciparum, Plasmodium vivax, Plasmodium ovale and Plasmodium malariae, propagated by the stings of some species of anopheles mosquitos.

Preferably, the compounds of formula (Ib) and (IaD) are useful for the treatment and/or prevention of cancers, bacterial infections and/or malaria.

Preferably, the compounds of formula (Ia) and (IaD) are useful for the treatment and/or prevention of malaria.

The present invention also concerns a compound of formula (Ia), (Ib), (IaD) or (II) such as defined in the invention for use thereof as medicinal product, preferably a compound of formula (Ib) for use thereof as medicinal product.

The present invention also concerns the use of a compound of formula (Ia), (Ib), (IaD) or (II) to prepare a medicinal product for use thereof in the treatment and/or prevention of a cancer, bacterial infection and/or malaria.

The present invention also concerns a method to treat and/or prevent cancers, bacterial infections and/or malaria comprising the administration to a patient of at least one compound of formula (Ia), (Ib), (IaD) and/or (II).

The present invention also concerns compositions, preferably pharmaceutical compositions, comprising at least one compound of formula (Ia), (Ib), (IaD) and/or (II).

The present invention therefore concerns pharmaceutical compositions comprising, as active ingredient, at least one compound of formula (Ia), (Ib) and/or (II) of the invention. These pharmaceutical compositions contain an efficient dose of at least one compound of formula (Ia), (Ib), (IaD) and/or (II) of the invention, or a pharmaceutical acceptable salt, and at least one pharmaceutically acceptable excipient. Said excipients are selected in accordance with pharmaceutical form and desired administration mode, from among usual excipients known to persons skilled in the art. The pharmaceutical compositions may contain other active compounds.

In the pharmaceutical compositions of the present invention for oral, sublingual, subcutaneous, intramuscular, intra-venous, topical, local, intratracheal, intranasal, transdermal or rectal administration, the active ingredient of formula (Ia), (Ib), (IaD) and/or (II) such as defined in the invention, or a salt thereof, can be administered in unit form in a mixture with conventional pharmaceutical excipients, to animals and to human beings for the treatment and/or prevention of the above-mentioned diseases.

Suitable unit administration forms comprise forms via oral route such as tablets, hard and soft capsules, powders, granules and oral solutions or suspensions; sublingual, per os, intratracheal, intraocular, intranasal via inhalation administration forms; topical, transdermal, subcutaneous, intramuscular or intravenous administration forms; rectal administration forms and implants. For topical application, the compounds of the invention can be used in solutions, emulsions, creams, gels, ointments or lotions, without any limitation in this respect.

The present invention also concerns a compound of formula (Ia), (Ib), (IaD) or (II) for the marking of an organelle, preferably a lysosome.

The present invention also concerns the use of a compound of formula (Ia), (Ib), (IaD) or (II) as marker or biomarker, e.g. of at least one organelle, preferably a lysosome.

The present invention also concerns a method to detect at least one lysosome, comprising the contacting of at least one cell preferably of eukaryote type, with a compound of formula (Ia), (Ib), (IaD) or (II).

The present invention also concerns a method for the marking of at least one lysosome comprising the contacting of at least one cell preferably of eukaryote type, with a compound of formula (Ia), (Ib), (IaD) or (II).

According to one particular embodiment, said detection method further comprises the following steps:
contacting living cells with at least one compound of formula (Ia), (Ib), (IaD) or (II);
detecting the possible presence of compounds of the invention in the cytosol, and more particularly in lysosomes.

According to one variant the detection method comprises the detection, by fluorescence recording means, of the possible presence of compounds of the invention.

According to one variant, the detection method comprises the detection of the possible presence of compounds of the invention by immunodetection.

According to one variant, the detection method comprises the detection of lysosomes and compounds of the invention.

Advantageously, the invention concerns a method to evaluate cell proliferation applying a detection method of the invention.

The invention also concerns a biological marker kit comprising at least one compound of formula (Ia), (Ib), (IaD) or (II). Advantageously, this kit also comprises at least one compound detectable by imaging e.g. a fluorophore compound or at least one antibody.

### DESCRIPTION OF THE FIGURES

FIGS. 1, 2 and 3 show the cytotoxic activity of marmycin A and of doxorubicin (intercalary agent used in the treatment of cancer).

FIG. 1 shows the percentage cell viability, as a function of the concentration in μM of marmycin A (solid line) or doxorubicin (dotted line), of U2OS cells (osteosarcoma cancer line).

FIG. 2 shows the percentage cell viability, as a function of the concentration in μM of marmycin A (solid line) or doxorubicin (dotted line), of A2780 cells (ovarian cancer line). The IC₅₀ values obtained are 9.8 μM for marmycin A and 0.06 μM for doxorubicin.

FIG. 3 shows the percentage cell viability, as a function of the concentration in μM of compound 33 (thick solid line), doxorubicin (dotted line) or etoposide (thin solid line) of HT1080 cells (fibrosarcoma line).

FIG. 4 shows the photographs obtained by fluorescence wherein: <<DAPI>> represents marking at the cell nucleus with DAPI (4′,6′-diamidino-2-phenylindole); <<Doxorubicin>> represents marking with doxorubicin at the cell nucleus; <<DAPI/Marmycin A>> represents marking with DAPI at the cells and marking with Marmycin A at the lysosomes.

FIG. 5 shows the photographs obtained by fluorescence wherein: <<Marmycin A>> represents the marking and location of Marmycin A; <<DND-22>> represents the marking and location of the lysosomes; <<Merge>> represents the colocation of Marmycin A with the lysosomes; <<ZOOM>> represents a photograph with magnification of a particular area of the photograph: <<Merge>> showing the colocation of Marmycin A and lysosomes.

FIG. 6 shows the photographs obtained with fluorescence wherein: <<DAPI>> represents marking with DAPI of the cell nucleus; <<GFP-Lamp1>> represents marking of lysosomes with the antibody anti-LAMP1; <<Marmycin A>> represents the marking and location of Marmycin A; <<MERGE>> represents the co-location of Marmycin A and GFP-Lamp1 lysosomal proteins at the lysosomes, and of DAPI at the cell nucleus.

FIG. 7 gives a Western Blot analysis where <<4 !>> represents Doxorubicin and <<1 !>> Marmycin A.

FIG. 8 shows the photographs obtained by fluorescence wherein: <<DAPI>> represents marking with DAPI at the cell nucleus; <<GFP-Lamp1>> represents marking of lysosomes with the antibody anti-LAMP1; <<Artesumycin>> represents the marking and location of artesumycin; <<MERGE>> represents the co-location of artesumycin and lysosomal proteins GFP-Lamp1 at the lysosomes, and of DAPI at the cell nucleus (scale 10 μm).

FIG. 9 shows the percentage of viable MDA-MB-231 cells after 72 h treatment with doxorubicin (DXR) (circles, thin dots), marmycin A (squares, dots), artesunate (circles, thick dots), artesumycin (circles, solid line) and a combination of marmycin A and artesunate (circles, dots and dashes).

FIG. 10 shows the cytotoxic activity of artesumycin (TC5) on a HMLER CD24-cancerous stem cell line.

FIG. 11 shows the cytotoxic activity of artesumycin (TC5) on a HMLER ID2 cancerous cell line

### EXAMPLES

### Example 1: Synthesis of Compounds of Formulas (V) and (VI) of the Invention

The synthesis of dienophile 5 (compound of formula (V)) is performed in accordance with Scheme 1. Compound 8 (supplier, Aldrich) is protected under conventional conditions in the form of diacetylated naphthalene 9 which is then brominated in an acid medium (see Kitani, Y.; Morita, A.; Kumamoto, T.; Ishikawa, T. Helvetica Chimica Acta 2002, 85, 1186 Carreno, M. C. et al Chem. Eur. J. 2000, 6, 906 Shis, C.; Swenton, J. S. J. Org. Chem. 1982, 47, 2825).

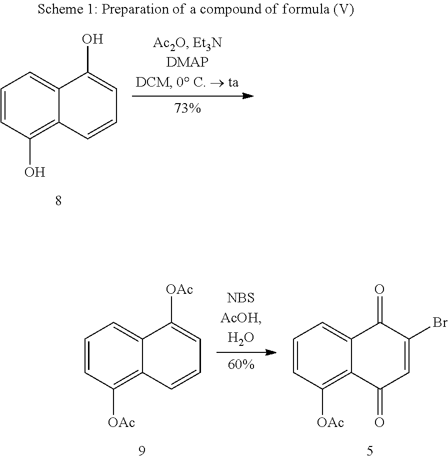 [SMILES: Oc1cccc2c(O)cccc12 ; CC(=O)Oc1cccc2c(OC(C)=O)cccc12 ; CC(=O)Oc1cccc2c1C(=O)C=C(Br)C2=O]

The diene 15 (compound of formula (VI)) is prepared from compound 10 that is commercially available (supplier, Aldrich) according to Scheme 2. A first bromination step is first carried out. Compound 11 is protected as an acetal 12 in the presence of ethylene glycol and a catalytic amount of acid. The reaction is conducted at a concentration of 0.4 M in under 2 hours under reflux with benzene. Compound 12 is converted to 13 via a palladium-catalysed coupling step to graft the vinyl chain thereupon.

Deprotection of 13 to 14 is obtained in a mild acid medium to prevent isomerisation of the double bond and the formation of an unsaturated α-β ketone. The action of methylmagnesium bromide in the presence of cerium chloride under cold conditions allows the racemic formation of the desired diene 15.

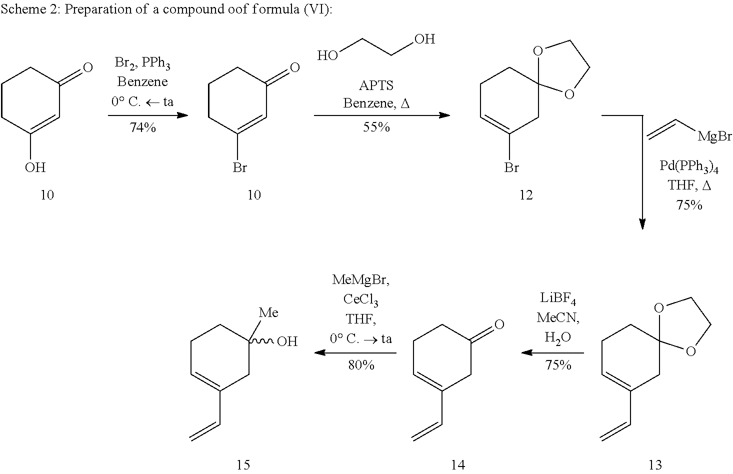

### Example 2: Coupling Step a and Preparation of a Formula (III) Compound of the Invention

Compound 16 (compound of formula (III′)) is prepared according to following Scheme 3:

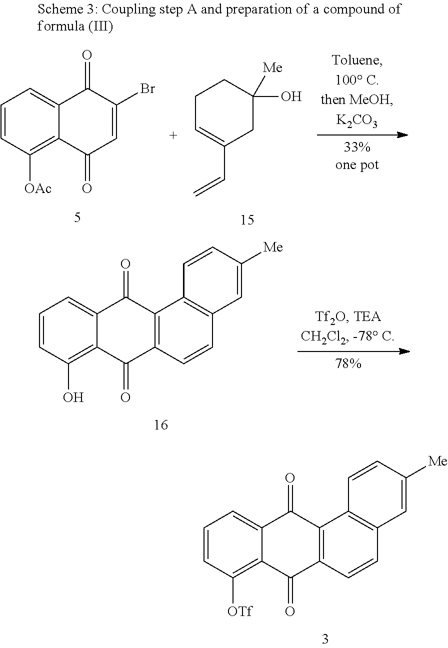

Compounds 5 and 15 are brought under reflux with toluene for 16 hours, after which the medium is subjected to slow evaporation for about 1 hour with a rotary evaporator in a bath at 60° C. The medium is re-dissolved in MeOH and left under agitation in the dark for 4 hours. 3 equivalents of potassium carbonate are then added to (compound of formula (III)).

### Example 3: Synthesis of Aminopyranose

Following the procedure described in the literature (cf. Raymond N. Russell, Theresa M. Weigel, Oksoo Han, Hung-wen Liu. Carbohydrate Research, Volume 201, Issue 1, 15 Jun. 1990, Pages 95-114 (b) Brimacombe, J. S.; Hanna, R.; Saeed, M. S.; Tucker, L. C. N. J. Chem. Soc., Perkin Trans. 1 1982, 2583-2587.) compound 4 is prepared from L-rhamnopyranose 7 (supplier, Alfa Aeser).

After protection under standard conditions with benzylidene at position 2 and 3, compound 17 is again protected via action of MOMCl (chloromethyl methyl ether) at position 4. The use of a strong base on compound 18 allows selective deprotection to form the ketone 19 at position 3. Compound 19 reacts with O-benzylhydroxylamine in the presence of sodium acetate to form the intermediate 20. In the presence of cerium chloride and methyllithium, compound 20 is selectively methylated to 21 which undergoes hydrogenolysis to obtain the desired product 4, in accordance with Scheme 4 below.

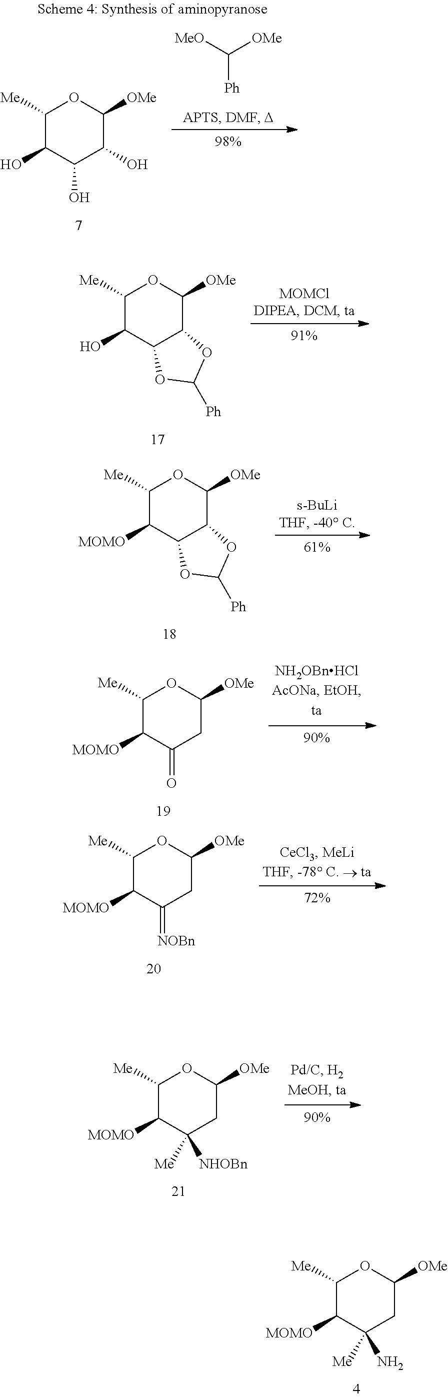

### Example 4: Coupling Step B, Preparation of Formula (II) Compounds of the Invention

### Example 4.1

Coupling B of the invention was conducted a first time according to following Scheme 5:

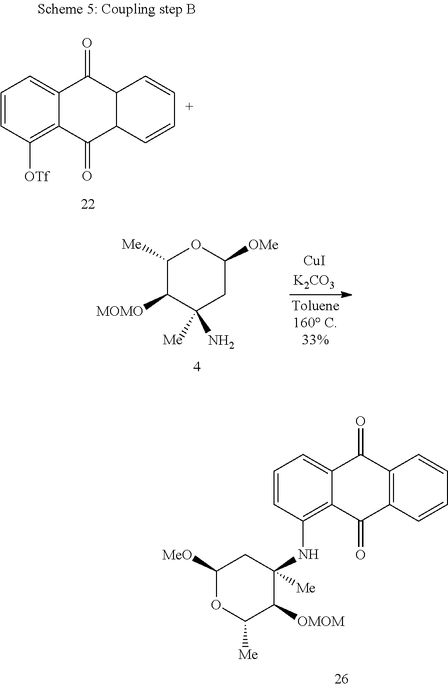

Coupling B was performed in the presence of K₂CO₃ in toluene under reflux with 20 mol % Cu—I, 2 eq. of K₂CO₃, 3 eq. of 4 in toluene at 160° C. for 72 hours. A yield of 33% was obtained.

Different operating conditions were also tested as shown in Table 1 below.

TABLE 1

| Catalyst | | Base | | | | |
|---|---|---|---|---|---|---|
| (mol %) | Equiv. 4 | (equiv.) | Solvent | T° C. | t (h) | Yield (%) |
| Cul 10 | 2 | K₂CO₃ 2 | Toluene | 140 | 72 | 33 |
| Cul 20 | 3 | K₂CO₃ 2 | Toluene | 140 | 24 | 22-32 |
| Cul 20 | 1 (2 eq | K₂CO₃ 2 | Toluene | 150 | 24 | 14 |
| | 23) | | | | | |
| Cul 20 | 3 | K₂CO₃ 2 | Toluene | 150 | 24 | 33 |
| Cul 20 | 2 | K₂CO₃ 3 | Toluene | 150 | 24 | 18 |
| Cul 10 | 3 | K₂CO₃ 1.4 | CH₃CN | 150 | 24 | 3 |
| Cul 20 | 5 | K₂CO₃ 2 | Toluene | 160 | 72 | 33 |

### Example 4.2

Coupling B of the invention was also performed according to following Scheme 6.

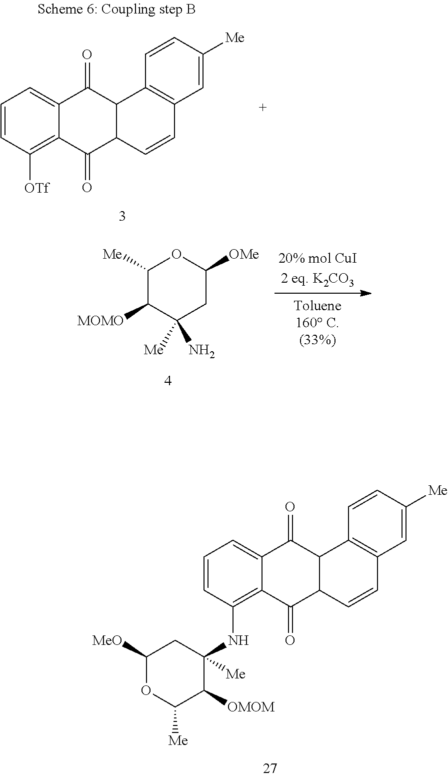

A yield of 33% was also obtained.

In a dry tube with septum the following were mixed under argon: triflate 3 (34.00 mg, 0.081 mmol), the catalyst CuI (3.08 mg, 0.016 mmol, 20% mol) and K₂CO₃ (22.3 mg, 0.162 mmol, 2 equiv.), after which the amine 4 was added (88.64 mg, 0.405 mmol, 5 equiv.) dissolved in 4 mL of dry toluene.

The septum was then replaced on the stopper under argon and the reaction left to take place at 160° C. for 72 h. The medium was cooled, washed in saturated NaHCO₃ solution (5 mL), and extracted with DCM (3×5 mL). The organic layers were dried over Na₂SO₄ and the solvent removed in vacuo. Purification by flash-chromatography on silica gel (heptane/AcOEt, 8:2) was finally carried out.

13.05 mg of compound 27 (33%, 8-(((2S,3R,4R,6R)-6-methoxy-3-(methoxymethoxy)-2,4-dimethyltetrahydro-2H-pyran-4-yl)amino)-3-methyltetraphene-7,12-dione) were obtained in the form of a purple solid.

¹H NMR (300 MHz, CDCl₃) δ 10.56 (s, 1H), 8.35 (d, J=7.5 Hz, 1H), 8.29-8.17 (m, 1H), 7.81-7.62 (m, 2H), 7.57 (d, J=7.1 Hz, 1H), 7.46 (t, J=8.0 Hz, 1H), 7.35 (d, J=8.6 Hz, 1H), 4.89 (q, J=6.8 Hz, 2H), 4.63 (d, J=3.2 Hz, 1H), 4.31 (dq, J=9.4, 6.2 Hz, 1H), 3.48 (s, 3H), 3.28 (d, J=9.4 Hz, 1H), 3.14 (s, 3H), 2.77 (d, J=14.7 Hz, 1H), 1.82 (dd, J=14.8, 4.2 Hz, 1H), 1.65 (s, 3H), 1.39 (d, J=6.3 Hz, 3H). ¹³C NMR (126 MHz, CDCl₃) b 187.33, 185.15, 151.03, 138.46, 137.16, 136.42, 135.50, 134.22, 134.20, 131.96, 128.82, 128.58, 128.45, 127.74, 123.06, 120.54, 115.62, 113.84, 99.09, 97.56, 87.49, 64.48, 56.81, 55.75, 54.90, 38.38, 26.54, 21.73, 18.70. HRMS (ESI-TOF) calculated for C₂₉H₃₂NO₆ [M+H]⁺ 490.2224; found: 490,2225.

### Example 5: Cyclisation Step Ca, Preparation of Formula (Ia) Compounds of the Invention

### Example 5.1

Compounds 30 and 31 were placed in the presence of 1.5 eq HBF₄ (50% w/w in aqueous solution) under reflux in CH₃CN. The cyclisation product 1 was obtained with a yield of 17%.

Therefore, in a single step, both cyclisation and MOM deprotection are carried out to obtain the natural product (Scheme 7).

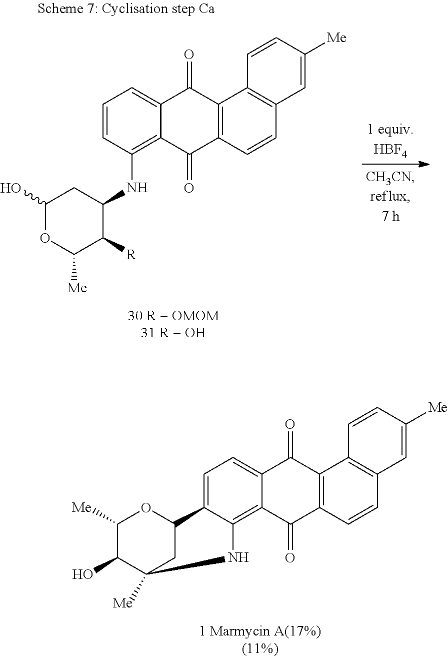

Several tests were carried out as shown in Table 2 below.

TABLE 2

| Acid | Equiv. | Solvent | T° C. | t (h) | Yield (%) |
|---|---|---|---|---|---|
| HBF₄OEt₂ | 1 | CH₃CN | reflux | 4 | 12 |
| HBF₄ 50% | 1.5 | CH₃CN | reflux | 24 | 7 |
| m/m | | | | | |
| aqueous sol. | | | | | |
| HBF₄ 50% | 1 | CH₃CN | reflux | 7 | 11 |
| m/m | | | | | |
| aqueous sol. | | | | | |
| HBF₄ 50% | 60 mol % | CH₃CN | reflux | 7 | 12 |
| m/m | | | | | |
| aqueous sol. | | | | | |
| HCl | 2 | CH₃CN | reflux | 8 | 8 |

In particular, with 60 mol % HBF₄ under reflux with acetonitrile, product 1 was obtained with a yield of 13%.

### Example 5.2

A solution of compound 27 (21.00 mg, 0.0429 mmol) and HBF₄ (5 μL, 0.0429 mmol, 1 equiv.) in 2 mL of CH₃CN was placed under reflux for 8 h.

The mixture was then cooled, diluted in dichloromethane (5 mL) and washed with saturated NaHCO₃ solution (3×5 mL). The organic phase was dried over Na₂SO₄ and the solvent evaporated in vacuo. Thin layer preparative chromatography using heptane/AcOEt (1:1) as eluent allowed 2.3 mg of Marmycin A to be obtained with a yield of 13%

Recrystallization by evaporation of a CH₂Cl₂:Heptane mixture allowed red crystal needles to be obtained.

¹H NMR (950 MHz, CDCl₃) δ 9.59 (s, 1H), 9.55 (d, J=8.8 Hz, 1H), 8.31 (d, J=8.5 Hz, 1H), 8.08 (d, J=8.5 Hz, 1H), 7.67 (s, 1H), 7.60 (d, J=7.3 Hz, 1H), 7.57 (dd, J=8.8, 1.7 Hz, 1H), 7.53 (d, J=7.4 Hz, 1H), 4.85-4.80 (m, 1H), 3.22 (t, J=9.4 Hz, 1H), 3.17 (dq, J=9.2, 6.0 Hz, 1H), 2.56 (s, 1H), 2.19 (ddd, J=13.2, 3.2, 1.2 Hz, 1H), 1.87 (dd, J=13.2, 1.8 Hz, 1H), 1.55 (s, 1H), 1.27 (d, J=6.0 Hz, 1H). ¹³C NMR (239 MHz, CDCl₃) δ 186.53, 185.77, 148.75, 138.81, 136.56, 136.47, 136.15, 134.72, 134.65, 132.21, 128.82, 128.54, 128.31, 127.80, 127.42, 122.34, 116.13, 111.35, 79.16, 69.36, 69.33, 51.76, 35.05, 25.03, 21.73, 18.43. HRMS (ESI-TOF) calculated for C₂₆H₂₄NO₄ [M+H]⁺ 414.1700; found: 414.1703.

### Example 6: Cyclisation Step Cb, Preparation of Formula (Ib) Compounds of the Invention

Compounds of formula (Ib) were also obtained from compounds 28 and 32. In a strong basic medium, the products 33 (oxamarmycin) and 34 were obtained with respective yields of 52% and 65%, as shown in Scheme 8 below.

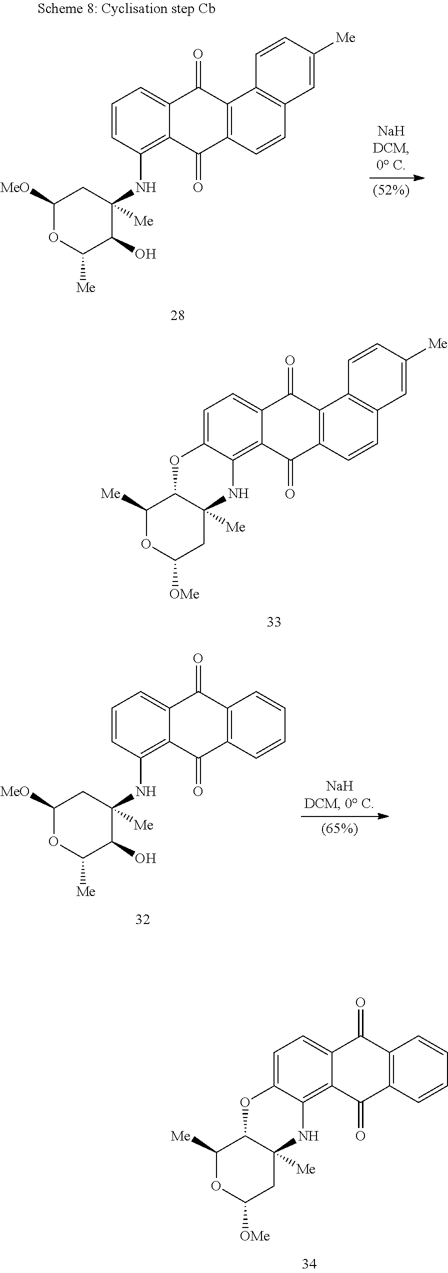 [SMILES: CO[C@H]1C[C@@](C)(Nc2cccc3c2C(=O)c2ccc4cc(C)ccc4c2C3=O)[C@@H](O)[C@H](C)O1 ; CO[C@H]1C[C@]2(C)Nc3c(ccc4c3C(=O)c3ccc5cc(C)ccc5c3C4=O)O[C@H]2[C@H](C)O1 ; CO[C@H]1C[C@@](C)(Nc2cccc3c2C(=O)c2ccccc2C3=O)[C@@H](O)[C@H](C)O1 ; CO[C@H]1C[C@]2(C)Nc3c(ccc4c3C(=O)c3ccccc3C4=O)O[C@H]2[C@H](C)O1]

A solution of compound 28 (5.00 mg, 0.0112 mmol) in 2 mL of dry DMF was cooled to 00° C. and added to NaH (2.70 mg, 0.1123 mmol, 10 equiv.):

after 2 h at this temperature, the reaction was halted with MeOH. The solvent was evaporated in vacuo and preparative thin layer chromatography using petroleum ether/AcOEt (8:2) as eluent allowed 2.54 mg of O-Marmycin to be obtained with a yield of 52% (purple solid).

¹H NMR (300 MHz, CDCl₃) δ 9.65 (d, J=8.9 Hz, 1H), 9.40 (s, 1H), 8.37 (d, J=8.6 Hz, 1H), 8.07 (d, J=8.7 Hz, 1H), 7.66 (s, 1H), 7.58 (m, 2H), 7.06 (d, J=8.4 Hz, 1H), 4.72 (d, J=4.3 Hz, 1H), 3.94 (dq, J=12.1, 6.1 Hz, 1H), 3.81 (dd, J=9.8, 1.3 Hz, 1H), 3.28 (s, 3H), 2.55 (s, 3H), 2.28 (d, J=14.8 Hz, 1H), 2.06 (dd, J=14.6, 4.7 Hz, 1H), 1.38 (s, 3H), 1.31 (d, J=6.2 Hz, 3H). ¹³C NMR (126 MHz, CDCl₃) δ 186.01, 185.61, 145.39, 138.58, 136.73, 136.58, 135.08, 134.08, 131.92, 129.62, 129.38, 128.90, 128.72, 127.77, 122.59, 119.66, 117.69, 112.88, 97.52, 79.61, 62.13, 55.58, 48.38, 41.89, 28.49, 21.72, 17.45. HRMS (ESI-TOF) calculated for C₂₇H₂₆NO₅ [M+H]⁺ 444.1805; found: 444.1826.

### Comparative Example 7: Coupling as Per Buchwald-Hartwig Reaction

### Example 7.1

Compound 22 was obtained under the conditions described in Scheme 3 from commercially available alcohol, and the amine 23 is also commercially available (supplier, Aldrich). The reaction was carried out according to the following Scheme:

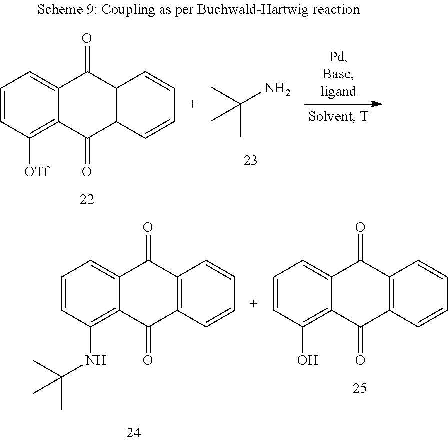

Different catalysts, ligands and solvents and different bases and temperatures were tested (cf. Table 3). In the best case, only 13% of product 24 was observed. The remainder was a hydrolysis product 25 and reagent 22 which had not reacted.

TABLE 3

| Catalyst | | Base | | | | Yield |
|---|---|---|---|---|---|---|
| (mol %) | Ligand | (equiv.) | Solvent | T° C. | t (h) | (%) |
| Pd(dba)₂^{a} | BINAP | NaO^{t}Bu | Toluene | 110 | 16 h | — |
| Pd(OAc)₂^{a} | BINAP | NaO^{t}Bu | Toluene | 110 | 17 h | traces |
| Pd(OAc)₂^{b} | DPPF | Cs₂CO₃ | Toluene | 110 | 20 h | — |
| [PdCl(allyl)]₂^{a} | DPPF | NaO^{t}Bu | Toluene | 110 | 20 h | — |
| Pd(OAc)₂^{c} | BINAP | Cs₂CO₃ | Toluene | 110 | 22 h | — |
| Pd(OAc)₂^{c} | BINAP | Cs₂CO₃ | CH₃CN | Reflux | 48 h | — |
| Pd(OAc)₂^{c} | BINAP | Cs₂CO₃ | DMF | Reflux | 8 h | traces |
| Pd(OAc)₂^{a} | P(Ph₃)₃ | Cs₂CO₃ | Toluene | 110 | 48 h | — |
| Pd(OAc)₂^{c} | BINAP | NaO^{t}Bu | THF | Reflux | 48 h | — |
| Pd(OAc)₂^{d} | BINAP | Cs₂CO₃ | DMF | 150 | 30 min | traces |
| Pd(OAc)₂^{d} | BINAP | Cs₂CO₃ | Toluene | 160 | 30 min | traces |
| Pd(OAc)₂^{e} | BINAP | NaO^{t}Bu | Toluene | 180 | 35 min | 13 |
| Pd(OAc)₂^{f} | BINAP | NaO^{t}Bu | Toluene | 110 | 35 min | 4 |

^{a}5% Pd, 5% L, 1.4 eq. base, 1.2 eq. LiCl
^{b}10% Pd, 15% L, 2 eq. Base, 2 eq. LiCl
^{c}5% Pd, 10% L, 2 eq. Base, 2 eq. LiCl
^{d}5% Pd, 10% L, 1 eq. base, 1 eq. LiCl
^{e}15% Pd, 15% L, 1.2 eq. base;
^{f}15% Pd, 15% L, 1.2 eq. base, 1.4 eq. LiCl.

### Example 7.2

Coupling was also performed between triflate 22 and aminopyranose 4. The yield dropped to 3% and much hydrolysis product 25 was observed (Scheme 10).

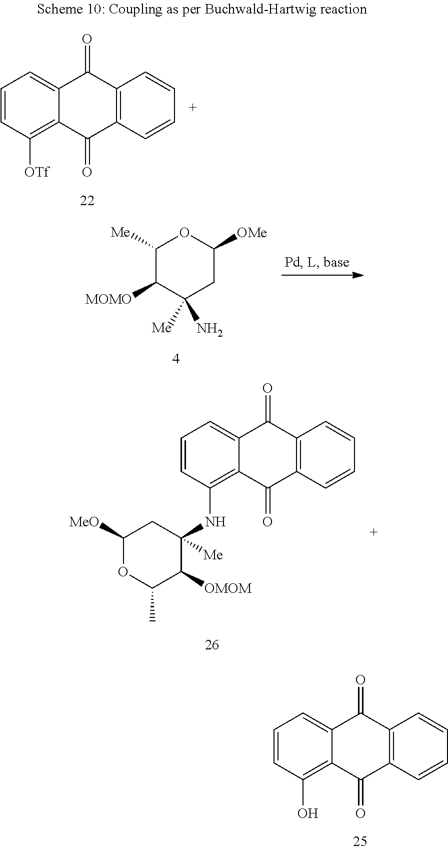

As a result, coupling B of the invention allows a yield of the formula (II) compound of at least 30% to be obtained, much higher than the yield obtained with coupling of Buchwald-Hartwig type.

### Comparative Example 8: Cyclisation Test Performed in the Presence of an Acid

The pathway of intramolecular C-glycosylation promoted by a Lewis or Brønsted acid was tested. An extensive study was conducted by varying the acid (BF₃ Et₂O, TMSOTf, ScOTf, InOTf, PPTS, APTS, Cp₂HfCl₂/AgClO₄) without any conclusive results. Only deprotection of compound 27 to 28, and the formation of amino-anthraquinone 29 were observed.

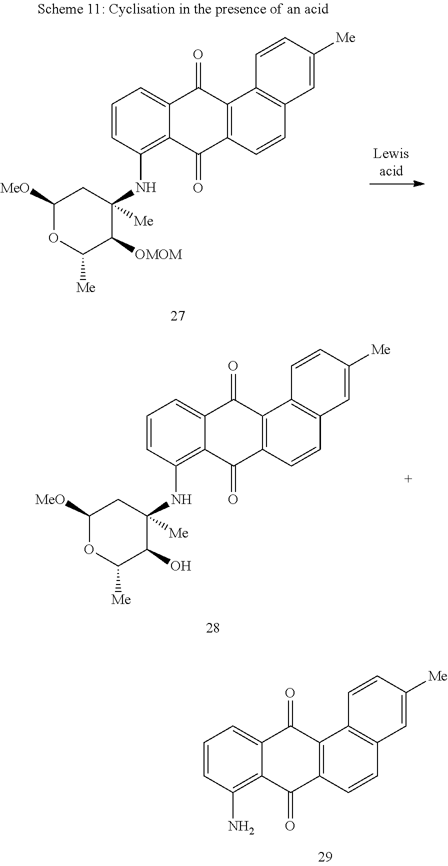

Contrary to the operation conditions for cyclisation Ca and Cb of the invention, cyclisation in the presence of an acid did not allow the desired compounds to be obtained.

### Example 9: Cytotoxic Activity of the Compounds of the Invention

Cell proliferation assays were carried out in the following manner to demonstrate the cytotoxic activity of the compounds of the invention:

U2OS and A2780 cells purchased from ATCC were held in McCoy's 5A or RPMI 1640 medium respectively, supplemented with 10% foetal bovine serum (FBS)) and 1× Antibiotic-Antimycotic (all from Gibco®) at 37° C. with 5% CO₂. Measurement of cell viability was carried out by seeding 2000 cells per well in a 96-well plate. N-Acetyl Cystein (NAC, A9165 Sigma) or Pan-caspase zVAD-FMK inhibitor (550377 by BD pharmingen) were pre-treated for 1 hour or 30 minutes respectively before treatment with Marmycin A. The reagent <<CellTiter-Blue®>> Reagent) (20 μl/well) was added after 24, 48, or 72 treatment hours, and the cells were incubated for one hour before fluorescence detection (560(20)Ex/590(10)Em) on a Perkin Elmer Wallac 1420 Victor2 Microplate Reader.

The same protocol was followed to evaluate cell viability of HT1080 cells in the presence of compound 33 of Example 6 (called Oxamethoxymarmycin), doxorubicin or etoposide.

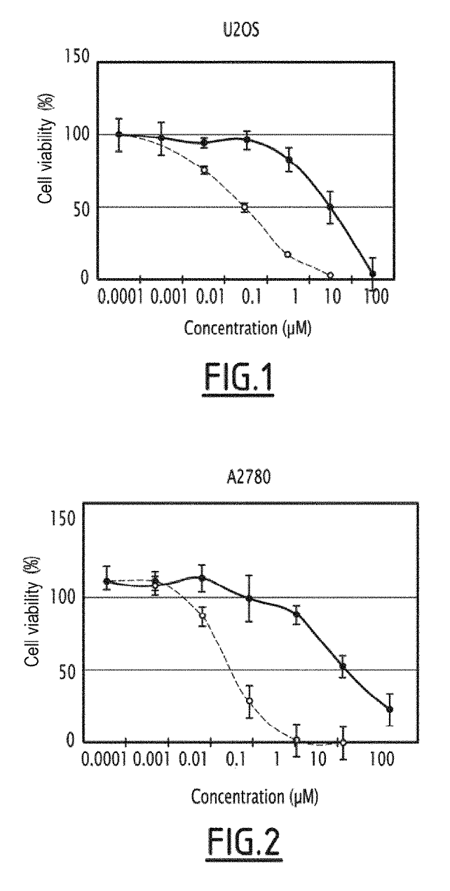

The results are given in FIGS. 1 to 3.

In FIG. 1, the IC₅₀ values obtained (<<IC₅₀>> representing the half-maximal inhibiting concentration of cells by the compound being evaluated) are 10.3 μM for marmycin A and 0.1 μM for doxorubicin, at 72 hours.

In FIG. 2, the IC₅₀ obtained are 9.8 μM for marmycin A and 0.06 μM for doxorubicin, at 72 hours.

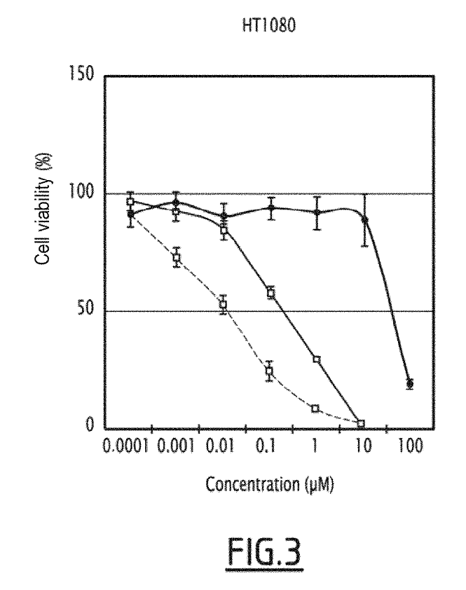

In FIG. 3, the IC₅₀ obtained are 75 μM for Oxamethoxymarmycin, 0.035 μM for doxorubicin and 0.5 μM for etoposide, at 72 hours.

### Example 10: Cell Location of the Compounds of the Invention

U2OS cells cultured to less than 40% confluence were treated for 24 hours with 10 μM of compound unless otherwise indicated. The marker LysoTracker® Blue DND-22 (L7525, Molecular Probes@) was added 30 minutes before fixing. GFP-Lamp1 was transiently transfected following the manufacturer's instructions. To summarise, 5 mL of CellLight® Lysosomes-GFP BacMam 2.0 (C10596, Life Technologies) were mixed with 200 μM of U2OS culture medium and added to each well of a 24-well plate. After overnight incubation (16 hours), the cells were washed and treated with Marmycin A as indicated in Example 9. For analysis by immunofluorescence, the cells were fixed 12 minutes in 2% formaldehyde/PBS, permeabilised 10 min with 0.1% Triton X/PBS and fixed for 1 h with 5% BSA, 0.2% Tween-20/PBS (blocking buffer). Cover glasses were incubated with anti-LAMP1, p62, antibodies, diluted to 1/100 in the blocking buffer overnight at 4° C. The cells were then washed 3 times with 0.2% Tween-20/PBS, and incubated as described above with anti-mouse Alexa 488-conjugated secondary antibody (A11029, Invitrogen) diluted to 1/500 in blocking buffer. The cover glasses were washed as described above and mounted with VectaShield® mounting medium for analysis by fluorescence with or without DAPI (Vector Laboratories Ltd). The images were obtained with a Leica microscope (Zeiss) and analysed with ImageJ software.

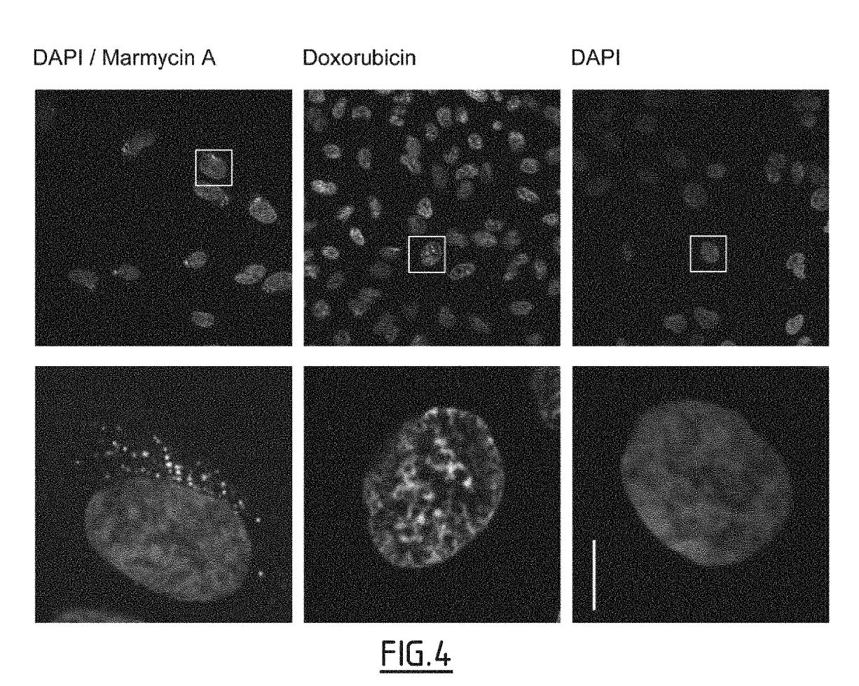

FIGS. 4 to 6 show the photographs obtained by fluorescence. FIG. 4 with the comparison of different photographs shows that Marmycin A is not located at the cell nucleus but at the site of the lysosomes.

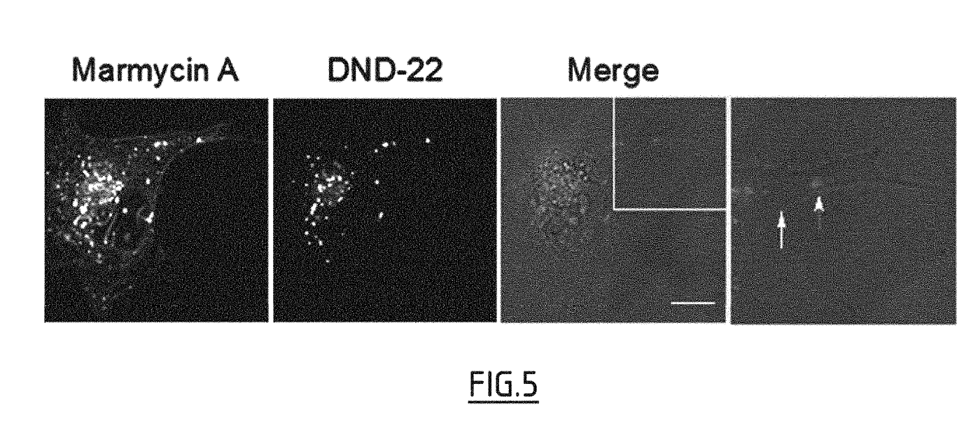

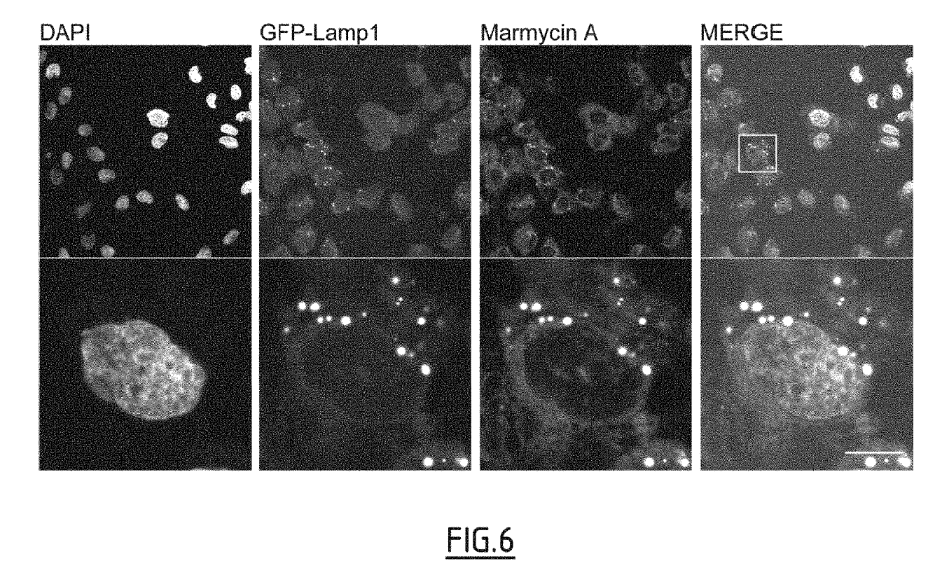

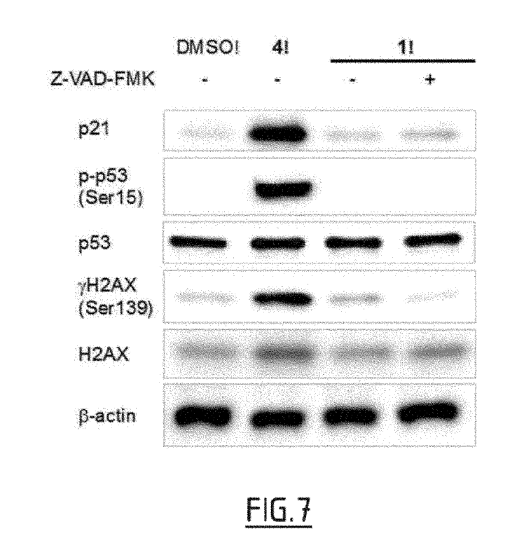

FIG. 5 shows the co-location of Marmycin A with the lysosomes. Marmycin A is therefore present and accumulates in the lysosomes.

FIG. 6 also shows that Marmycin A accumulates in the lysosomes.

### Example 11: Western Blot

The cells treated as indicated in Example 10 were washed twice in PBS and lysed with Laemmli buffer 2×. The cell extracts were brought to the boil for five minutes at 100° C. and quantified with a Nanodrop 2000 device (Thermal Scientific). 100 μg of protein lysate were separated with 4-20% Mini-PROTEAN® TGX Stain-Free™ gel (BioRad) and transferred onto a nitrocellulose membrane (Amersham). Stains were detected with different antibodies such as anti-β-actin (ab8226, Abcam), anti-p62 (610833, BD Transduction Laboratories™), anti-H2AX (PA1-14198, Thermal Scientific), anti-gH2AX (Ser139) (#2577, Cell Signaling), anti-p21, anti-p53 (1C12) (#2524, Cell Signaling), anti-p-p53, anti-LC3B (#2775, Cell Signaling), anti-BID, diluted to 1/1000 in 5% BSA, 0.1% Tween-20/TBS.

Western Blot analysis specifically showed that Marmycin A (<<1 !>>) induces a response that only scarcely damages caspase-dependent DNA, which tallies with the absence of direct marking of the genome, whereas Doxorubicin (<<4 !>>) generates a DNA-damage response (phosphorylation of H2AX on ser139, phosphorylation of P53, induction of P21).

### Example 12: Preparation of Formula (IaD) Compounds, Addition Step D of the Invention

To a mixture of marmycin A (2.3 mg, 0.0055 mmol), artesunate (2.1 mg, 0.0056 mmol) and 4-dimethylaminopyridine (DMAP) (0.7 mg, 0.0006 mmol, 10 mol %) in dry dichloromethane (0.3 mL) the addition was made of N,N′-dicyclohexylcarbodiimide solution (1.1 mg, 0.0055 mmol) in dichloromethane (0.2 mL) at 0° C. The resulting solution was mixed overnight at ambient temperature and concentrated to a dry residue.

The reaction product was purified by thin layer chromatography (heptane/AcOEt, 1:1) to obtain a red solid called artesumycin (1.8 mg, 42%).

¹H NMR (500 MHz, CDCl₃) δ 9.65 (s, 1H), 9.59 (d, J=9.0 Hz, 1H), 8.39 (d, J=8.5 Hz, 1H), 8.11 (d, J=9.0 Hz, 1H), 7.69 (s, 1H), 7.63-7.56 (m, 2H), 7.50 (d, J=7.5 Hz, 1H), 5.80 (d, J=9.5 Hz, 1H), 5.41 (s, 1H), 4.83 (s, 1H), 4.79 (d, J=9.5 Hz, 1H), 3.44 (dq, J=12.0, 6.0 Hz, 1H), 2.79-2.74 (m, 4H), 2.58-2.54 (m, 4H), 2.36 (td, J=14.0, 4.0 Hz, 1H), 2.25 (dd, J=13.0, 2.0 Hz, 1H), 2.02 (ddd, J=7.5, 4.0, 3.0 Hz, 1H), 1.89-1.86 (m, 2H), 1.75 (dd, J=13.5, 4.0 Hz, 2H), 1.67 (dd, J=13.0, 3.0 Hz, 1H), 1.62-1.58 (m, 2H), 1.44-1.41 (m, 4H), 1.42 (s, 3H), 1.35 (dd, J=13.0, 3.0 Hz, 1H), 1.09 (d, J=6.0 Hz, 3H), 1.95-1.02 (m, 1H), 0.93 (d, J=6.0 Hz, 3H), 0.86 (d, J=7.0 Hz, 3H). ¹³C NMR (151 MHz, CDCl₃) b 186.6, 185.6, 172.1, 171.2, 148.6, 138.7, 136.5, 135.9, 134.9, 134.6, 132.2, 128.8, 128.6, 128.3, 127.8, 126.7, 122.5, 115.9, 111.0, 104.6, 92.3, 91.5, 80.2, 79.6, 69.3, 66.4, 51.5, 51.1, 45.2, 37.3, 36.2, 35.0, 34.1, 31.9, 29.8, 29.1, 28.9, 26.1, 25.1, 24.6, 22.1, 21.7, 20.3, 18.0, 12.2. HRMS (ESI-TOF) calculated for C₄₅H₄₉NNaO₁₁⁺ [M+Na]⁺ 802.3198; found: 802.3204. [α]_{D}²⁰+253 (c 0.015, tetrahydrofuran).

### Example 13: Cell Location of Formula (IaD) Compounds of the Invention

U2OS cells cultured to less than 40% confluence were treated for 24 hours with 10 μM artesumycin. The marker LysoTracker® Blue DND-22 (L7525, Molecular Probes®) was added 30 minutes before fixing. GFP-Lamp1 was transiently transfected following the manufacturer's instructions. To summarise, 5 mL of CellLight® Lysosomes-GFP BacMam 2.0 (C10596, Life Technologies) were mixed with 200 μM of U2OS culture medium and added to each well of a 24-well plate. After overnight incubation (16 hours) the cells were washed and treated with artesumycin as indicated in Example 9. Immunofluorescence analysis was carried out in the same manner as in Example 10.

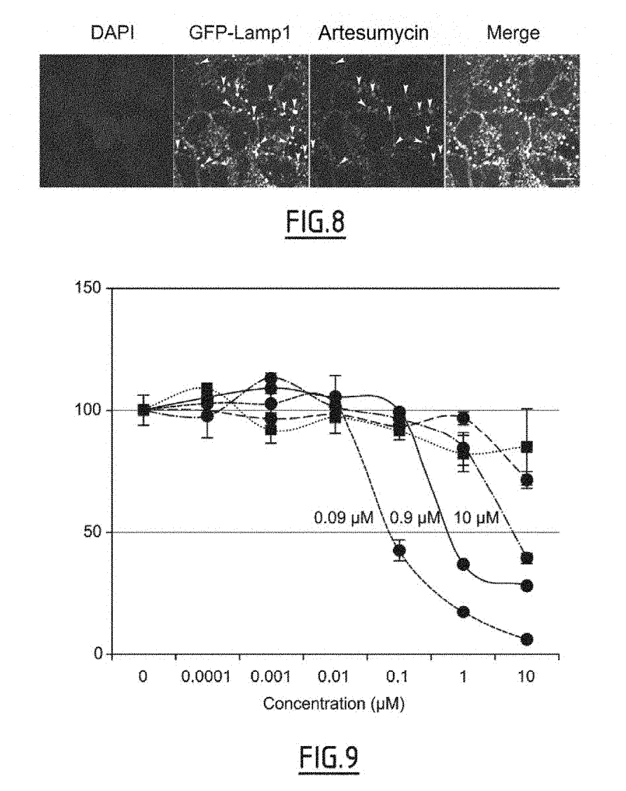

FIG. 8 gives photographs obtained by fluorescence and illustrates the co-location of artesumycin with the lysosomes. Artesumycin is therefore present and accumulates in the lysosomes.

### Example 14: Cytotoxic Activity of the Formula (IaD) Compounds of the Invention

Cell proliferation assays were performed in similar manner to those in Example 9 to demonstrate the cytotoxic activity of the formula (IaD) compounds of the invention.

MDA-MB-231 cells (human breast cancer cells) purchased from ATCC were held in McCoy's 5A medium, supplemented with 10% foetal bovine serum (FBS)) and 1× Antibiotic-Antimycotic (all from Gibco®) at 37° C. with 5% CO₂.

Cell viability was measured by seeding 2000 cells per well in a 96-well plate. N-Acetyl Cystein (NAC, A9165 Sigma) or Pan-caspase zVAD-FMK inhibitor (550377 from BD pharmingen) were pre-treated for 1 hour or 30 minutes respectively before treatment with the assayed compounds. The reagent <<CellTiter-Blue® Reagent>> (20 μl/well) was added after a treatment time of 24, 48 or 72 hours, and the cells were incubated for one hour before fluorescence detection (560(20)Ex/590(10)Em) on a Perkin Elmer Wallac 1420 Victor2 Microplate Reader.

The assayed compounds were doxorubicin, marmycin A, artesunate, artesumycin and a combination of marmycin A and artesunate (cf. FIG. 9).

FIG. 9 shows the cytotoxic action of marmycin A and artesumycin on a cancer cell line. Artesumycin at a concentration of 0.9 μm allows cell viability of less than 50% to be obtained. The cytotoxic activity of artesumycin is higher than that of marmycin A and also higher than that of marmycin A combined with artesunate. The IC₅₀ of artesumycin is 0.9 μM. The IC₅₀ of the artesunate-marmycin A combination is 10 μM.

A synergic effect is therefore observed for artesumycin compared with the activity of the marmycin A and artesunate combination.

### Example 15: Cytotoxic Activity of the Formula (IaD) Compounds of the Invention

Cell viability: cell viability was assessed using the following protocol:

Seeding 1000 cells/well in a 96-well plate. The cells were treated for 72 hours. After a treatment time of 72 hours the reagent CellTiter-Blue® Reagent (G8081, Promega) was added and the cells incubated for one hour before recording fluorescence intensities (Excitation, 560/20 nm; Emission, 590/10 nm) using a Perkin Elmer Wallac 1420 Victor2 Microplate Reader.

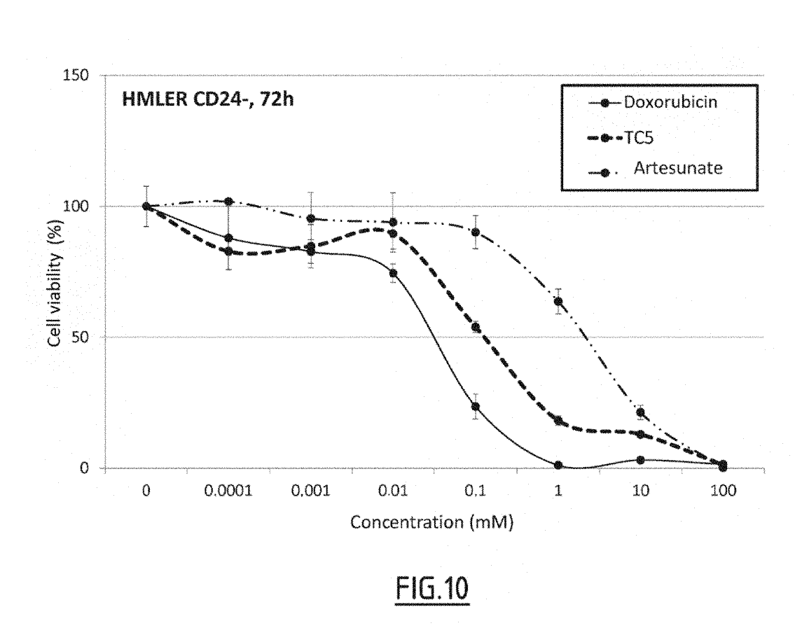

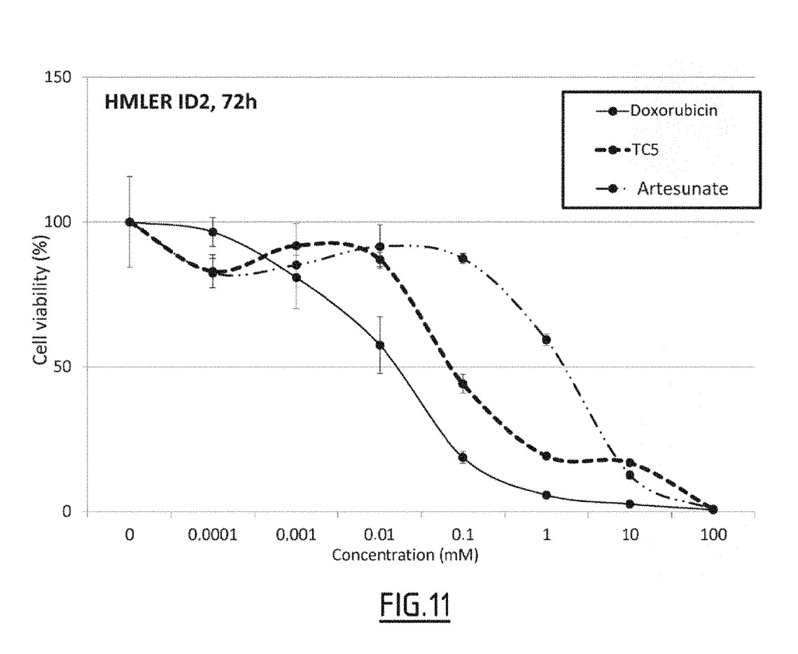

The results are given in FIGS. 10 and 11 for different cell cultures.

Cell culture: the following material was used: saline buffer <<Dulbecco's Phosphate-Buffered Saline>> (14190-094, 500 mL, Gibco), DMEM/F12 (31331-028, 500 mL, Gibco), DMEM high-glucose with UltraGlutamine (BE12-604F/U1, BioWhittaker, Lonza), McCoy's 5A medium (Modified) (26600-023, Gibco), RPMI 1640 with L-glutamine (BE12-702F/U1, BioWhittaker, Lonza), Foetal Bovine Serum—FBS, 10270-106, Gibco), Hydrocortisone (H0888, Sigma), Insulin (10516, Sigma or 19278, Sigma), BD Epidermal growth factor human recombinant (hEGF, 354052, BD Biosciences), PEN-STREP (DE17-602E, BioWhittaker, Lonza), puromycin dihydrochloride (A11138-02, 460 Life Technologies).

The human mammary epithelial cell line was infected with a retrovirus carrying hTERT, SV40 and the oncogenic allele HrasV12, called HMLER CD44high/CD24 low cells, not expressing E-cadherin and Vimentin (reference HMLER CD24low or HMLER CD44+/CD24−), courteously offered by A. Puisieux (INSERM).

The line referenced HMLER ID2 is a hTert, SV40, HRasV12 transformed line, isogenic but non-stem.

The HMLER CD44^{high}/CD24^{low} cells (Cancer stem cells—CSCs), HMLER CD44^{high}/CD24^{high} (non-CSCs) were cultured in DMEM/F12 supplemented with 10% FBS, 10 μg/mL insulin, 0.5 μg/mL hydrocortisone, 10 ng/mL hEGF and 0.5 μg/mL of puromycin.

A mycoplasma assay was performed using a mycoplasma PCR detection kit (G238, 470 Applied Biological Materials) confirming the absence of cell contamination.

The assayed compounds were doxorubicin, artesunate and artesumycin (TC5) (cf. FIGS. 10 and 11).

FIG. 10 shows the cytotoxic activity of artesumycin on a cancerous stem cell line HMLER CD24−. Artesumycin at a concentration of 1 μm allows cell viability well below 50% to be obtained (about 18%). The cytotoxic activity of artesumycin is higher than that of artesunate. The IC₅₀ of artesumycin is 100 nM.

FIG. 11 shows the cytotoxic activity of artesumycin on a HMLER ID2 cancerous cell line. Artesumycin at a concentration of 0.1 μm allows cell viability well below 50% to be obtained (about 44%). The cytotoxic activity of artesumycin is higher than that of artesunate. The IC₅₀ of artesumycin is 98 nM.

These results illustrate the efficacy of artesumycin on human malignant cell lines resistant to conventional therapies such as taxol, whether they be stem or non-stem.

1. A method for preparing a compound of formula (II): 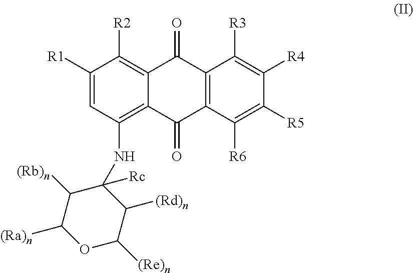
where R₁, R₂, R₃, R₄, R₅, R₆, Ra, Rb, Rc, Rd and Re are each independently an atom or group of atoms,
n represent the number of Ra, Rb, Rd and Re radicals and are equal to 2;
said method comprising a coupling step B of a compound of formula (III): 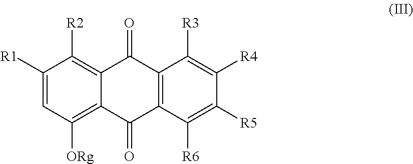
where the radicals R₁ to R₆ are such as defined for the formula (II) compound and Rg is a reactive group with the NH₂ group of the compound of formula (IV),
with a compound of formula (IV): 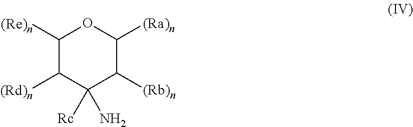
where the radicals Ra to Re and n are such as defined for the formula (II) compound, said coupling step being conducted in the presence of a copper-containing compound.

2. The method according to claim 1, characterized in that the coupling step B is conducted in the presence of a copper-containing compound of formula Cu—X, X being selected from the group formed by Cl, Br, I, SO₄ and OAc.

3. The method according to claim 1, characterized in that the coupling step B is conducted in the presence of 5 mol % to 25 mol %, of copper-containing compound.

4. The method according to claim 1, characterized in that coupling is conducted in the presence of a base e.g. K₂CO₃.

5. The method according to claim 1, further comprising a coupling step A of a compound of formula (V): 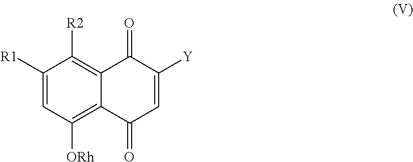
where R₁ and R₂ are such as defined in claim 1, Y being a reactive group with the carbon atom carrying the R₃ group of the formula (VI) compound and Rh being a protective group,
with a compound of formula (VI): 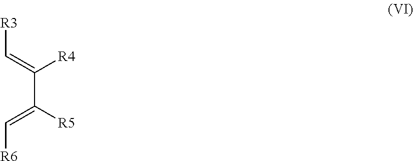
to obtain a compound of formula (III′): 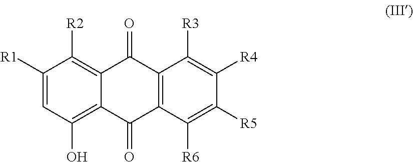
where the radicals R₁ to R₆ are such as defined in claim 1, in the presence of toluene or xylene.

6. A method for preparing a pentacyclic structure, comprising:
preparation of a formula (II) compound according to claim 1, and
a cyclisation step C of said formula (II) compound.

7. A method for preparing a compound of following formula (Ia): 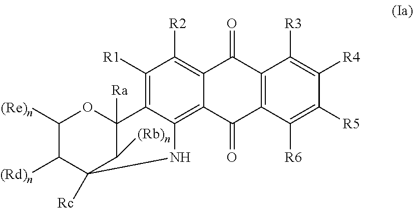
where the radicals R₁ to R₆ and Ra to Re and n are such as defined in claim 1, said method comprising a cyclisation step Ca via C—C glycosylation of a formula (II) compound such as obtained according to claim 1, in the presence of HBF₄ to form the compound of formula (Ia).

8. A method for preparing a compound of following formula (Ib): 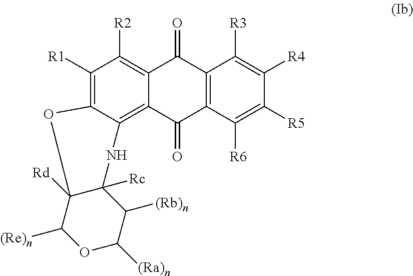
where the radicals R₁ to R₆ and Ra to Re and n are such as defined in claim 1, said method comprising a cyclisation step Cb via formation of an O—C bond of a compound of formula (II) such as obtained according to claim 1, in a basic medium.

9. A method for preparing a compound of formula (IaD) such as defined below, said method comprising an addition step D of a compound of formula (Ia): 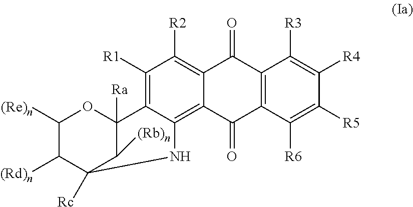
where the radicals R₁ to R₆, Ra to Re and n are such as defined in claim 1, and wherein at least one of the radicals Ra to Re is an OH group,
with a compound of following formula:
Rx-L-Rz (Ie)
where:
Rx is a reactive group allowing reaction with an OH function;
L is a group of atoms called a <<spacer group>>; and
Rz is an optionally substituted (C₃-C₂₀)heterocycle;
said addition step D resulting in the formation of a compound of following formula (IaD): 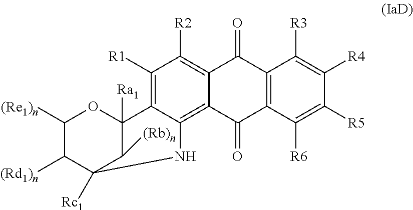
where the radicals R₁ to R₆ and n are such as defined in claim 1, and wherein the radicals Ra1 to Re1 have the same definitions as the radicals Ra to Re defined for formula (Ia) respectively,
among which at least one of the radicals Ra1 to Re1 represents the group resulting from the reaction between the groups OH and Rx, bonded covalently to L-Rz.

10. The method according to claim 1, wherein:
R₁, R₂, R₃, R₄, R₅ and R₆ are each independently selected from the group formed by: H, OH, halogen, C(O)OH, ═O, (C₁-C₁₀)alkyl, O(C₁-C₁₀)alkyl, OC(O)(C₁-C₁₀)alkyl, (C₂-C₁₀)alkenyl, C(O)O(C₁-C₁₀)alkyl, NH₂, NH(C₁-C₁₀)alkyl, N[(C₁-C₁₀)alkyl]₂, NHC(O)(C₁-C₁₀)alkyl, N(C₁-C₁₀)alkyl-C(O)(C₁-C₁₀)alkyl, C(O)NH₂, C(O)N(C₁-C₁₀)alkyl, C(O)N[(C₁-C₁₀)alkyl]₂, oses and epoxy groups, wherein said alkyls and/or said oses can be substituted; or
R₁ with R₂ and/or R₃ with R₄ and/or R₄ with R₅ and/or R₅ with R₆, together with the carbon atoms to which they are attached, form a (C₃-C₁₀)cycloalkyl or (C₆-C₁₀)aryl group, said cycloalkyl or aryl groups optionally being substituted, and wherein at least one of the carbon atoms may optionally be replaced by a heteroatom;
the radicals Ra are each independently selected from the group formed by: H, OH, halogen, C(O)OH, ═O, (C₁-C₁₀)alkyl, O(C₁-C₁₀)alkyl, OC(O)(C₁-C₁₀)alkyl, (C₂-C₁₀)alkenyl, C(O)O(C₁-C₁₀)alkyl, NH₂, NH(C₁-C₁₀)alkyl, N[(C₁-C₁₀)alkyl]₂ NHC(O)(C₁-C₁₀)alkyl, N(C₁-C₁₀)alkyl-C(O)(C₁-C₁₀)alkyl, C(O)NH₂, C(O)N(C₁-C₁₀)alkyl, C(O)N[(C₁-C₁₀)alkyl]₂, and the reactive groups allowing the formation of a glycosidic C—C bond, wherein said alkyls can be substituted;
the radicals Rb, Rc and Re are each independently selected from the group formed by: H, OH, halogen, C(O)OH, ═O, (C₁-C₁₀)alkyl, O(C₁-C₁₀)alkyl, OC(O)(C₁-C₁₀)alkyl, (C₂-C₁₀)alkenyl, C(O)O(C₁-C₁₀)alkyl, NH₂, NH(C₁-C₁₀)alkyl, N[(C₁-C₁₀)alkyl]₂, NHC(O)(C₁-C₁₀)alkyl, N(C₁-C₁₀)alkyl-C(O)(C₁-C₁₀)alkyl, C(O)NH₂, C(O)N(C₁-C₁₀)alkyl and C(O)N[(C₁-C₁₀)alkyl]₂, wherein said alkyls can be substituted;
the radicals Rd are each independently selected from the group formed by: H, OH, halogen, C(O)OH, ═O, (C₁-C₁₀)alkyl, O(C₁-C₁₀)alkyl, OC(O)(C₁-C₁₀)alkyl, (C₂-C₁₀)alkenyl, C(O)O(C₁-C₁₀)alkyl, NH₂, NH(C₁-C₁₀)alkyl, N[(C₁-C₁₀)alkyl]₂, NHC(O)(C₁-C₁₀)alkyl, N(C₁-C₁₀)alkyl-C(O)(C₁-C₁₀)alkyl, C(O)NH₂, C(O)N(C₁-C₁₀)alkyl, C(O)N[(C₁-C₁₀)alkyl]₂, (C₁-C₁₀)alkyl-COO⁻, methoxymethyloxygen, and the reactive groups allowing the formation of an O—C bond such as AcO, wherein said alkyls can be substituted.

11. A compound of following formula (Ia): 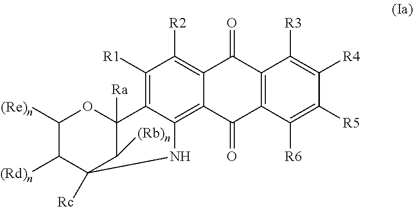
where the radicals R₁ to R₆ and Ra to Re and n are such as defined in claim 1, with the exception of the compounds where R₃ and R₄, together with the carbon atoms to which they are attached, form a phenyl group, and
with the exception of the following compounds:

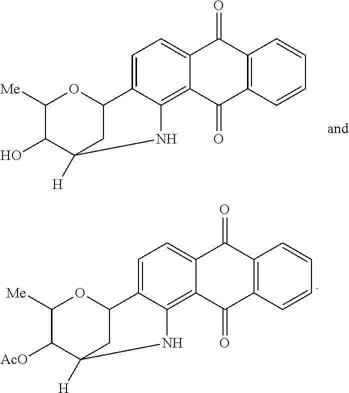 [SMILES: CC1OC2CC(Nc3c2ccc2c3C(=O)c3ccccc3C2=O)C1O ; CC(=O)OC1C2CC(OC1C)c1ccc3c(c1N2)C(=O)c1ccccc1C3=O]

12. A compound of following formula (Ib): 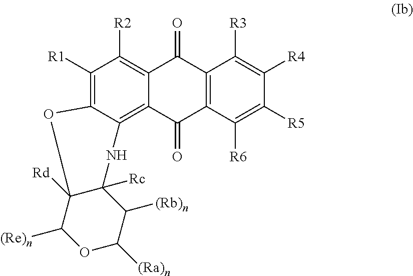
where the radicals R₁ to R₆ and Ra to Re and n are such as defined in claim 1.

13. A compound of following formula (IaD): 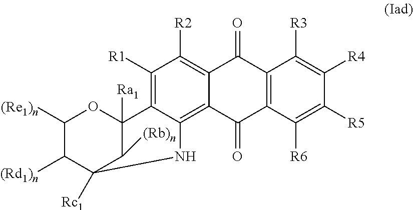
where the radicals R₁ to R₆ are each independently an atom or group of atoms, and n represents the number of Ra, Rb, Rd and Re radicals and are equal to 2, and the radicals Ra₁ to Re₁ are such as defined in claim 9.

14. A compound of following formula (IaD):

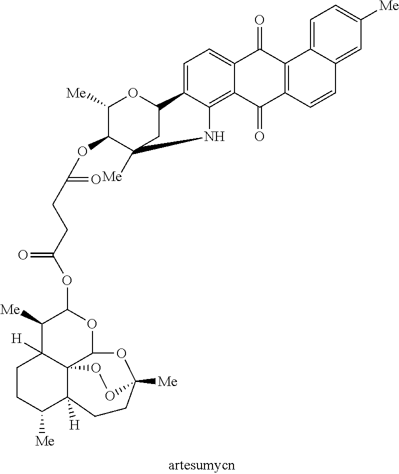

15. A pharmaceutical composition comprising a compound of formula (Ia), (Ib), (IaD) or (II) such as defined in claim 1.

16. (canceled)

17. (canceled)

18. (canceled)

19. A method for treating and/or preventing a disease selected among a cancer, bacterial infection and malaria, comprising administering to a mammal in need thereof a therapeutically effective amount of a compound of formula (Ia), (Ib), (IaD) or (II) such as defined in claim 1.

20. A method for treating and/or preventing a resistant cancer, in particular a cancer having cancerous stem cells, comprising administering to a mammal in need thereof a therapeutically effective amount of a pharmaceutical composition comprising a compound of formula (Ia), (Ib), (IaD) or (II) such as defined in claim 1.

21. A method to detect at least one lysosome, comprising the contacting of at least one cell, with a compound of formula (Ia), (Ib), (IaD) or (II) such as defined in claim 1.

22. A method for the marking of at least one lysosome comprising the contacting of at least one cell preferably of eukaryote type, with a compound of formula (Ia), (Ib), (IaD) or (II) such as defined in claim 1.
